(19) United States
(12) Patent Application Publication
(10) Pub. No.: US 2025/0066338 A1
(43) Pub. Date: Feb. 27, 2025

(54) CERTAIN 3-AZABICYCLO[3.1.0]HEXANES AS GLP-1 RECEPTOR MODULATORS

(71) Applicant: AstraZeneca AB, Sodertalje (SE)

(72) Inventors: Magnus POLLA, Sodertalje (SE); Joakim BERGMAN, Sodertalje (SE); Johan SUNDELL, Sodertalje (SE); Jonas BRÅNALT, Sodertalje (SE); Johan KAJANUS, Sodertalje (SE); Ekaterina RATKOVA, Sodertalje (SE); Magnus JOHANSSON, Sodertalje (SE)

(21) Appl. No.: 18/718,844

(22) PCT Filed: Dec. 15, 2022
(86) PCT No.: PCT/EP2022/086079

§ 371 Date: Jun. 12, 2024

Related U.S. Application Data
(60) Provisional application No. 63/265,496, filed on Dec. 16, 2021.

(51) Int. Cl. C07D 405/14; A61K 31/4439

(52) U.S. Cl. CPC C07D 405/14; A61K 31/4439

(57) ABSTRACT

There are disclosed certain 3-azabicyclo[3.1.0]hexanes, and pharmaceutically acceptable salts thereof, together with compositions containing them and their use in therapy. The compounds are GLP-1 receptor modulators and are thereby particularly useful in the treatment or prophylaxis of cardiovascular disease and metabolic conditions, for example Type 2 diabetes.

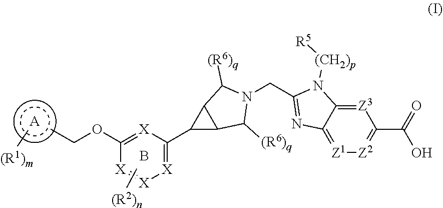

### TECHNICAL FIELD

The technical field relates to certain 3-azabicyclo[3.1.0]hexanes, to their use in the treatment of cardiovascular disease and metabolic conditions, for example type 2 diabetes, and to pharmaceutical compositions containing them.

### BACKGROUND

Obesity and type 2 diabetes (T2D) are major and growing health problems worldwide (Lancet, 2014, 9922, 1068-1083). The two diseases are strongly associated with each other, with obesity proceeding development of insulin resistance and T2D. T2D is associated with several comorbidities including cardiovascular disease, renal disease, hypertension, stroke, non-alcoholic fatty liver disease (NAFLD) and non-alcoholic steatohepatitis (NASH) (Lancet, 2005, 9468, 1415-1428).

Incretin hormones including GLP-1 (glucagon-like peptide-1) and GIP (glucose-dependent insulinotropic polypeptide) are gut peptides that are secreted after nutrient intake and stimulate insulin secretion (Diabetes Obes Metab., 2018, 20(Suppl.1), 5-21). GLP-1 secretion from the gut is impaired in obese subjects which may indicate a role in the pathophysiology of obesity (Regulatory Peptides, 2004, 122, 209-217).

GLP-1 is secreted from the L-cells in the lower gut in response to food intake. GLP-1 stimulates insulin secretion from the pancreatic □-cells, in a glucose dependent manner (Diabetologia, 1993, 36, 741-744). GLP-1 also inhibits glucagon secretion, reduces appetite and slows down gastric emptying. The GLP-1 receptor is also present in the heart, kidneys and immune system and activation has been shown to reduce blood pressure, increase natriuresis and decrease inflammation.

GLP-1 is a 37-amino acid peptide, post-translationally processed from pro-glucagon, a 158 amino acid precursor polypeptide (www.uniprot.org, pro-glucagon entry P01275). Several other peptides are also derived from proglucagon and processed in a tissue specific manor, including glucagon and oxyntomodulin. GLP-1 has very short half-life in vivo as it is rapidly degraded by dipeptidyl peptidase-4 (DPP-IV) (Front. Endocrinol. 2019, 10, Article 260, 1-10).

Incretin-based glucose- and body weight-lowering medications include GLP-1 receptor agonists, DPP-IV inhibitors and more recently also combinations of GLP-1 agonists and glucose-dependent insulinotropic polypeptide (GIP) agonists (Peptides, 2020, 125, Article 170202). Traditionally GLP-1 analogues are peptide hormones which have been modified to minimize DPP-IV cleavage and are administered as injectables. The first oral GLP-1 peptide was recently approved but bioavailability is low and the drug needs to be administered in the fasting state, 30 min before nutrient intake which may limit patient compliance (JAMA, 2017, 318(15), 1460-1470). The injectable peptides show increased efficacy over the oral peptides but are limited by the route of administration. Small molecule GLP-1 receptor agonists are in development from several companies and are expected to provide a therapeutic benefit versus peptide based therapies due to early use in the treatment paradigm.

Pharmacological stimulation of GLP-1 receptors has been shown to significantly reduce HbA1c levels, provide long term weight loss and reduce blood pressure. GLP-1 receptor agonists have also been shown to reduce cardiovascular events and prolong life in high-risk patients with T2D and are therefore recommended by the European Association for the Study of Diabetes (EASD) and American Diabetes Association (ADA) in patients with multiple risk factors of cardiovascular disease (CVD) independent of the patients glycemic control (Diabetes Care, 2020, 43, 487-493).

There remains a need for an easily-administered prevention and/or treatment for cardiometabolic and associated diseases.

WO2018/109607 discloses 6-carboxylic acids of benzimidazoles and 4-aza-, 5-aza-, 7-aza- and 4,7-diazabenzimidazoles as GLP-1 receptor agonists, processes to make said compounds, and methods comprising administering said compounds to a mammal in need thereof.

WO2019/239319 and WO2019/239371 disclose 6-carboxylic acids of benzimidazoles and 4-aza-, 5-aza- and 7-aza-benzimidazoles as GLP-1 receptor agonists, processes to make said compounds, and methods comprising administering said compounds to a mammal in need thereof.

WO2020/103815 disclose GLP1 receptor agonist compounds and pharmaceutical compositions thereof, for use in e.g. treating type 2 diabetes mellitus, pre-diabetes, obesity, non-alcoholic fatty liver disease, non-alcoholic steatohepatitis and cardiovascular disease.

WO2020/207474 disclose GLP1 receptor agonist compounds and pharmaceutical compositions thereof, for use in e.g. treating type 2 diabetes mellitus, pre-diabetes, obesity, non-alcoholic fatty liver disease, non-alcoholic steatohepatitis and cardiovascular disease.

WO2020/234726 disclose combinations of GLP-1 receptor agonist compounds and pharmaceutical compositions thereof and an acetyl-CoA carboxylase (ACC) inhibitor or a diacylglycerol acyltransferase (DGAT2) inhibitor, or a ketohexokinase (KHK) inhibitor or farnesoid X receptor (FXR) agonist, for use in e.g. treating type 2 diabetes mellitus, pre-diabetes, obesity, non-alcoholic fatty liver disease, non-alcoholic steatohepatitis and related diseases.

WO2020/263695 discloses glucagon-like peptide-1 receptor agonists and therapeutic uses of the compounds to treat type II diabetes mellitus.

WO2021/081207 discloses compounds that bind to and act as agonists or modulators of the glucagon-like peptide-1 receptor (GLP-1R) and act as agonists or modulators of GLP-1R. The disclosure further relates to the use of the compounds for the treatment and/or prevention of diseases and/or conditions by said compounds.

WO2021/018023 discloses compounds for modulating a Glucagon-like peptide-1 (GLP-1) receptor, and a pharmaceutical use thereof.

WO2021/096284 and WO2021/096304 discloses compounds that act as GLP-1 receptor agonists, for use as therapeutic agents for metabolic diseases.

WO2021/112538 discloses compounds which serves as a GLP-1 receptor agonist and may be useful in the prevention or treatment of a disease associated with GLP-1 activity.

WO2021/154796 discloses GLP-1R agonists, and compositions, methods, and kits thereof. Such compounds are generally useful for treating a GLP-1R mediated disease or condition.

WO2021/160127 discloses GLP-1 agonists, pharmaceutical compositions, and methods of use thereof.

WO2021116874 discloses of solid forms of 2-[[4-[(S)-2-(5-chloropyridin-2-yl)-2-methylbenzo[d][1,3]dioxol-4-yl]piperidin-1-yl]methyl]-1-[[(S)-oxetan-2-yl]methyl]-1H-benzo[d]imidazole-6-carboxylic acid, 1,3-dihydroxy-2-(hydroxymethyl)propan-2-amine salt for pharmaceutical use.

CN113493447A discloses a compound that can be used as a GLP-1 receptor agonist. WO2021197464 discloses fused imidazole derivatives, preparation methods and medical use as a therapeutic agent, especially as GLP-1 receptor agonists.

CN113480534A discloses benzimidazole or azabenzimidazole-6-carboxylate compound that can activate GLP-1R downstream signaling pathway.

WO2021154796 discloses compounds as GLP-1R agonists, and compositions, methods, and kits thereof.

WO2021219019 discloses GLP-1 agonists of formula I, including pharmaceutically acceptable salts and solvates thereof, pharmaceutical compositions, and methods of using the same.

WO2021244645 discloses five-membered heteroaromatic imidazole compounds I and their medical use.

WO2021249492 discloses methyl-substituted benzobisoxazole compound and the use thereof in the preparation of drugs for treating related diseases.

CN113816948A discloses fused imidazole derivatives as GLP-1 receptor agonist in the treatment of diabetes.

WO2021254470 discloses preparation of 6-oxo-3,6-dihydropyridine derivative and a pharmaceutical composition containing the derivative, are used as therapeutic agents, in particular as GLP-1 receptors agonist and in the preparation of drugs for the treatment and/or prevention of diabetes.

WO2022007979 discloses a fused imidazole derivative, a preparation method therefor, a pharmaceutical composition containing the derivative, and the use of same as a therapeutic agent, in particular the use thereof as a GLP-1 receptor agonist.

CN113831337A discloses heterocyclic nitrogen compounds as GLP-1 receptor agonist.

WO2022068772 discloses a kind of benzimidazole derivative, its preparation method and application as GLP-1R agonists.

WO2022042691 discloses GLP-1 agonists, including pharmaceutically acceptable salts and solvates thereof, and pharmaceutical compositions including the same.

WO2022040600 discloses compounds that may be used as a glucagon-like peptide-1 receptors (GLP-1R) agonist.

WO2022028572 discloses GLP-1 agonists, including pharmaceutically acceptable salts and solvates thereof, and pharmaceutical compositions including the same.

WO2022031994 discloses compounds and pharmaceutical compositions thereof, for use in, e.g. treating type 2 diabetes mellitus, pre-diabetes, obesity, non-alc. fatty liver disease, non-alc. steatohepatitis, and cardiovascular disease.

CN114591308A discloses piperazine-imidazole containing GLP-1R receptor agonist compounds and application thereof.

WO2022111624 discloses benzimidazole derivatives that are agonists of a glucagon-like peptide-1 receptor (GLP-1R).

WO2022109182 discloses polyheterocyclic benzimidazole compounds and their preparation and use in the treatment of GLP-1R mediated diseases.

CN114478497A discloses a kind of aryl alkyl acid GLP-1 receptor agonist, its preparation method and application in treatment or prevention of GLP-1-mediated diseases and related diseases.

WO2022078380 discloses compounds that are GLP-1 agonists.

WO2022078407 discloses compounds that are GLP-1 agonists.

WO2022078152 discloses a kind of benzimidazolone compounds, their preparation method and application as GLP-1 receptor agonist.

CN114716423A discloses 5,6-dihydro-1,2,4-triazine compounds as GLP-1 receptor agonist.

CN114634510A discloses imidazolopyridine derivatives, which can be used to prepare drugs for treating GLP-1 receptor agonist mediated diseases.

CN114591296A discloses aromatic heterocyclic derivatives as GLP-1R agonists.

WO2022192430 discloses GLP-1R agonists and compositions, methods, and kits thereof.

WO2022192428 discloses GLP-1R agonists and compositions, methods, and kits thereof.

WO2022184849 discloses GLP-1R agonists, uses and pharmaceutical compositions thereof.

CN114907351A discloses tricyclic GLP-1 receptor agonists.

WO2022165076 discloses substituted benzimidazolecarboxylic acids which are GLP-1 receptor modulator compounds.

CN114805336A discloses fused imidazole compounds that are GLP-1 receptor agonists.

CN114763352A discloses benzimidazole derivatives and its application as GLP-1 receptor agonist.

WO2022199458 discloses thiophene GLP-1 receptor agonist compounds.

WO2022199661 discloses compounds that modulates the activity of GLP-1 receptor.

WO2022202864 discloses compounds that has GLP-1 receptor agonist activity.

WO2022216094 discloses compounds that has GLP-1 receptor agonist activity.

WO2022219495 discloses compounds that are activators of GLP-1.

WO2022235717 discloses benzimidazoyl GLP-1 receptor agonists.

WO2022225914 discloses carboxy-benzimidazole GLP-1 modulators.

WO2022225941 discloses carboxy-benzimidazole GLP-1 modulators.

J. Med. Chem. 2022, 65, 12, 8208-8226 discloses A Small-Molecule Oral Agonist of the Human Glucagon-like Peptide-1 Receptor.

Cell Research 2020, (39), 1140-1142 discloses structural insights into the activation of GLP-1R by a small molecule agonist.

An object is to provide novel GLP-1 receptor modulators useful in therapy. A further object is to provide novel compounds having improved safety profile, e.g with regards to selectivity for the GLP-1 receptor over e.g. phosphodiesterase 3 (PDE3) and/or having improved metabolic stability in the body.

### SUMMARY

There is provided compounds that are modulators of the glucagon-like peptide-1 (GLP1) receptor, their use as medicaments, pharmaceutical compositions containing them and synthetic routes to their production.

In one embodiment, there is provided a compound of Formula (I),

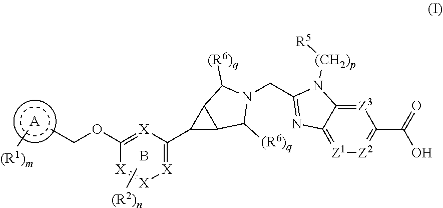

wherein
A is phenyl or pyridyl;
X is independently N or C, provided that no more than two atoms in the aromatic ring B are N;
Z¹ is N or CR³;
Z² and Z³ are each independently N or CR⁴, provided that when Z¹ or Z³ is N, Z² is CR⁴;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
R⁶ is independently selected from F, C₁₋₂alkyl and OC₁₋₂alkyl, wherein said C₁₋₂alkyl is substituted by 0, 1, 2 or 3 substituents independently selected from F;
m is 0, 1, 2 or 3;
n is 0 or 1;
p is 1, 2 or 3;
q is 0, 1 or 2;
or a pharmaceutically acceptable salt thereof.

The compounds of Formula (I) are modulators of the GLP-1 receptor. Thus, the compounds of Formula (I) can be used as a medicament, in particular for disorders, disease or conditions responsive to modulation of the GLP-1 receptor, and more specifically cardiovascular disease and metabolic conditions.

In another embodiment there is provided a compound of Formula (I), or a pharmaceutically acceptable salt of a compound of Formula (I), wherein the stereochemistry is undefined, e.g. a racemate or a mixture of diastereomers.

In another embodiment there is provided a compound of Formula (I), or a pharmaceutically acceptable salt of a compound of Formula (I), wherein the stereochemistry is defined.

In another embodiment there is provided a pharmaceutical formulation comprising a therapeutically effective amount of a compound of Formula (I), or a pharmaceutically acceptable salt of a compound of Formula (I), and a pharmaceutically acceptable diluent, excipient and/or inert carrier.

In a further embodiment there is provided a pharmaceutical formulation comprising a compound of Formula (I), or a pharmaceutically acceptable salt of a compound of Formula (I), for use in the treatment of a condition where modulation of the GLP-1 receptor would be beneficial.

In a further embodiment there is provided a compound of Formula (I), or a pharmaceutically acceptable salt of a compound of Formula (I), for use in therapy, especially in the treatment of cancer in a mammal, particularly a human.

In a further embodiment there is provided the use of a compound of Formula (I), or a pharmaceutically acceptable salt of a compound of Formula (I), for the manufacture of a medicament for the treatment of cardiovascular disease and metabolic conditions.

According to another aspect there is provided a process for the preparation of compounds of Formula (I), or pharmaceutically acceptable salts of compounds of Formula (I), and the intermediates used in the preparation thereof.

The compounds of Formula (I) described herein have the advantage that they may be more efficacious, be less toxic, be more selective, be more potent, produce fewer side effects, be more easily absorbed, and/or have a better pharmacokinetic profile (e.g. higher oral bioavailability and/or lower clearance), than compounds known in the prior art.

### DETAILED DESCRIPTION

This detailed description and its specific examples, while indicating embodiments, are intended for purposes of illustration only. Therefore, there is no limitation to the illustrative embodiments described in this specification. In addition, it is to be appreciated that various features that are, for clarity reasons, described in the context of separate embodiments, also may be combined to form a single embodiment. Conversely, various features that are, for brevity reasons, described in the context of a single embodiment, also may be combined to form sub-combinations thereof.

Listed below are definitions of various terms used in the specification and claims.

It is to be understood that where in this specification a group is qualified by “defined above” the said group encompasses the first occurring and broadest definition as well as each and all of the other definitions for that group.

In this specification, the term “modulator” is used to describe a compound that exhibit varying receptor agonism, either full agonism, or partial agonism.

It is to be understood that in this specification “C₁₋₄” means a carbon group having 1, 2, 3 or 4 carbon atoms.

It is to be understood that in this specification “C₁₋₂” means a carbon group having 1 or 2 carbon atoms.

In this specification, unless stated otherwise, the term “alkyl” includes both straight and branched chain alkyl groups and may be, but is not limited to, methyl, ethyl, n-propyl, i-propyl, n-butyl, sec-butyl, iso-butyl or tert-butyl.

It is to be understood that in this specification “(5- to 6-membered)heteroaryl” means an aromatic ring with 5 to 6 atoms and containing one or more heteroatoms independently selected from nitrogen, oxygen or sulphur.

It is to be understood that in this specification “(6-membered)heteroaryl” means an aromatic ring with 6 atoms and containing one or more heteroatoms independently selected from nitrogen, oxygen or sulphur.

It is to be understood that in this specification “(6-membered)heteroaryl” means for example pyridine.

It is to be understood that in this specification “(5-membered)heteroaryl” means an aromatic ring with 5 atoms and containing one or more heteroatoms independently selected from nitrogen, oxygen or sulphur.

It is to be understood that in this specification “(4- to 6-membered)heterocycloalkyl” means a partially or completely saturated ring system with 4 to atoms and wherein at least one of the ring carbon atoms is replaced with a heteroatom independently selected from nitrogen, oxygen or sulphur.

It is to be understood that in this specification a “heterocycloalkyl” substituent may be attached via a nitrogen atom having the appropriate valences, or via any ring carbon atom.

It is to be understood that in this specification a “heterocycloalkyl” or “heteroaryl” substituent may be further substituted, for example by a substituent selected from C₁₋₂alkyl.

In this specification, unless stated otherwise, the term “pharmaceutically acceptable” is used to characterize a moiety (e.g. a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

There is provided compounds of Formula (I) wherein X, Z¹, Z², Z³, R¹-R⁶, m, n, p and q are as defined in Formula (I).

In one embodiment X is independently N or C, provided that no more than two atoms in the aromatic ring B are N.

In a further embodiment X is N.

In still a further embodiment X is N or C.

In one embodiment Z¹ is N or CR³.

In a further embodiment Z¹ is N.

In still a further embodiment Z¹ is CR³.

R³ is selected from H, F, Cl, N(CH₃)₂, C₁₋₂alkyl and OC₁₋₂alkyl, wherein said C₁₋₂alkyl is substituted by 0, 1, 2 or 3 F.

In one embodiment Z² and Z³ are each independently N or CR⁴, provided that when Z¹ or Z³ is N, Z² is CR⁴.

In a further embodiment Z¹ and Z² are N.

In still a further embodiment Z and Z³ are N.

In still a further embodiment Z² and Z³ are N.

In still a further embodiment Z is N, Z² and Z³ are CR⁴.

In still a further embodiment Z² is N, Z and Z³ are CR⁴.

In still a further embodiment Z³ is N, Z and Z² are CR⁴.

In still a further embodiment Z¹, Z² and Z³ are CR⁴.

R⁴ is independently selected from H, F, Cl, OH, CH₃, CFH₂, CF₂H, CF₃, OCH₃, OCFH₂, OCF₂H and OCF₃.

In one embodiment R¹ is 0, 1, 2 or 3 substituents independently selected from F, Cl, Br, CN, OCH₃, OCFH₂, OCF₂H, OCF₃, CH₃, CFH₂, CF₂H and CF₃.

In a further embodiment R¹ is 0, 1 or 2 substituents independently selected from F, Cl, Br, CN, OCH₃, OCFH₂, OCF₂H, OCF₃, CH₃, CFH₂, CF₂H and CF₃.

In still a further embodiment R¹ is 0, 1, 2 or 3 substituents independently selected from F, Cl, Br, CN, OCH₃.

In still a further embodiment R¹ is 0, 1 or 2 substituents independently selected from F, Cl, Br, CN, OCH₃.

In still a further embodiment R¹ is 0, 1 or 2 substituents independently selected from F, Cl and CN.

In still a further embodiment R¹ is 0 or 1 substituents selected from F, Cl and CN.

In one embodiment R² is selected from 0 or 1 F, Cl or CN.

In one embodiment R³ is selected from H, F, Cl, N(CH₃)₂, C₁₋₂alkyl and OC₁₋₂alkyl, wherein said C₁₋₂alkyl is substituted by 0, 1, 2 or 3 F.

In a further embodiment R³ is selected from H, F, Cl, C₁₋₂alkyl and OC₁₋₂alkyl, wherein said C₁₋₂alkyl is substituted by 0, 1, 2 or 3 F.

In still a further embodiment R³ is selected from H, F, Cl, CH₃, CFH₂, CF₂H, CF₃, OCH₃, OCFH₂, OCF₂H and OCF₃.

In still a further embodiment R³ is selected from H, F, Cl, CH₃ and OCH₃.

In one embodiment R⁴ is independently selected from H, F, Cl, OH, CH₃, CFH₂, CF₂H, CF₃, OCH₃, OCFH₂, OCF₂H and OCF₃.

In a further embodiment R⁴ is independently selected from H, F, Cl, OH, CH₃ and OCH₃.

In still a further embodiment R⁴ is independently selected from H, F, Cl, CH₃ and OCH₃.

In still a further embodiment R⁴ is independently selected from H, F and Cl.

In one embodiment R⁵ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F.

In a further embodiment R⁵ is selected from C₁₋₄alkyl, O(C₁₋₄alkyl) and S(C₁₋₄alkyl), wherein said C₁₋₄alkyl is substituted by 0 or 1 substituent selected from CN or OCH₃, and 0, 1, 2 or 3 F.

In still a further embodiment R⁵ is selected from cyclopropyl, cyclobutyl, O(cyclopropyl) or S(cyclopropyl), said cyclopropyl and cyclobutyl is substituted by 0 or 1 substituent selected from CN, OCH₃, OCFH₂, OCF₂H, OCF₃ and CH₂CN and 0, 1, 2 or 3 F.

In still a further embodiment R⁵ is selected from (4- to 6-membered)heterocycloalkyl and (5- to 6-membered)heteroaryl, wherein said (4- to 6-membered)heterocycloalkyl and (5- to 6-membered)heteroaryl is substituted by 0 or 1 substituent selected from C₁₋₂alkyl.

In still a further embodiment R⁵ is selected from (5- to 6-membered)heteroaryl, wherein said (5- to 6-membered)heteroaryl is substituted by 0 or 1 substituent selected from C₁₋₂alkyl.

In still a further embodiment R⁵ is selected from (4- to 6-membered)heterocycloalkyl, wherein said (4- to 6-membered)heterocycloalkyl is substituted by 0 or 1 substituent selected from C₁₋₂alkyl.

In still a further embodiment R⁵ is oxetan-2-yl.

In one embodiment R⁶ is independently selected from F, C₁₋₂alkyl and OC₁₋₂alkyl, wherein said C₁₋₂alkyl is substituted by 0, 1, 2 or 3 substituents independently selected from F.

In a further embodiment R⁶ is independently selected from F, C₁₋₂alkyl and OC₁₋₂alkyl.

In still a further embodiment R⁶ is independently selected from F, CH₃ and OCH₃.

In one embodiment m is 0, 1, 2 or 3.

In a further embodiment m is 0, 1, or 2.

In still a further embodiment m is 1 or 2

In still a further embodiment m is 0 or 1.

In still a further embodiment m is 1.

In still a further embodiment m is 0.

In one embodiment n is 0 or 1.

In a further embodiment n is 1.

In still a further embodiment n is 0.

In one embodiment p is 1, 2 or 3.

In a further embodiment p is 1 or 2.

In still a further embodiment p is 1.

In one embodiment q is 0, 1 or 2.

In a further embodiment q is 0 or 1.

In still a further embodiment q is 0.

In one embodiment, there is provided a compound of Formula (Ia),

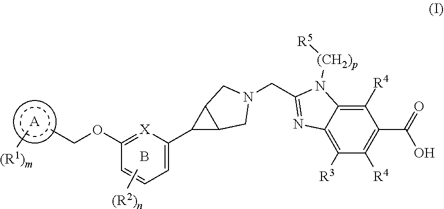

wherein
A is phenyl or pyridyl;
X is N or C;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said OC₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1, 2 or 3;
n is 0 Or 1;
p is 1, 2 or 3;
q is 0, 1 or 2;
or a pharmaceutically acceptable salt thereof.

In a further embodiment, there is provided a compound of Formula (Ia), wherein
A is phenyl;
X is N;
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1 or 2;
n is 0 or 1;
p is 1, 2 or 3;
or a pharmaceutically acceptable salt thereof.

In still a further embodiment, there is provided a compound of Formula (Ia),
wherein
A is phenyl;
X is N;
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said OC₁₋₂alkyl is substituted by 0, 1, 2 or 3 F;
R⁴ is independently selected from H, F, Cl, OH, CH₃ and OCH₃;
R⁵ is selected from (4- to 6-membered)heterocycloalkyl wherein said (4- to 6-membered)heterocycloalkyl is substituted by 0 or 1 substituent selected from C₁₋₂alkyl and;
m is 0, 1 or 2;
n is 0 or 1;
p is 1;
or a pharmaceutically acceptable salt thereof.

In one embodiment the compounds of Formula (I) are selected from:
2-(((1R,5S,6S)-6-(6-((4-Cyano-2-fluorobenzyl)oxy)-3-fluoropyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)pyridin-2-yl)-3-azabicyclo[3.1.0]hexan-3-yl)methyl)-1-(((S)-tetrahydrofuran-2-yl)methyl)-1H-benzo[d]imidazole-6-carboxylic acid,
2-(((1R,5S,6r)-6-(6-((4-Cyano-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylic acid,
4-Chloro-2-(((1R,5S,6S)-6-(6-((4-cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)pyridin-2-yl)-3-azabicyclo[3.1.0]hexan-3-yl)methyl)-4-methyl-1-(((S)-oxetan-2-yl)methyl)-1H-benzo[d]imidazole-6-carboxylic acid,
2-(((1R,5S,6r)-6-(6-((4-Cyano-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylic acid,
2-(((1R,5S,6S)-6-(6-((4-Chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6r)-6-(6-((4-Chloro-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylic acid,
2-(((1R,5S,6S)-6-(6-((4-Chloro-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid,
2-(((1R,5S,6r)-6-(6-((4-Chloro-2-fluorobenzyl)oxy)pyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylic acid,
2-(((1R,5S,6S)-6-(6-((4-Chloro-2-fluorobenzyl)oxy)pyridin-2-yl)-3-azabicyclo[3.1.0]hexan-3-yl)methyl)-4-fluoro-1-(((S)-oxetan-2-yl)methyl)-1H-benzo[d]imidazole-6-carboxylic acid,
2-(((1R,5S,6S)-6-(6-((4-Chloro-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylic acid,
2-(((1R,5S,6r)-6-(6-((4-Chloro-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylic acid,
2-(((1R,5S,6S)-6-(3-Chloro-6-((4-chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((4-Chloro-2-fluorobenzyl)oxy)-3-methylpyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((2,4-Difluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((2-Fluoro-5-methylbenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((2-(Difluoromethoxy)benzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
4-Methoxy-2-(((1R,5S,6S)-6-(6-((4-methylbenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid,
   and pharmaceutically acceptable salts thereof.

It shall be noted that any one of these specific compounds may be disclaimed from any of the herein mentioned embodiments.

In one embodiment there is provided a process for the preparation of compounds of Formula (I), or pharmaceutically acceptable salts of compounds of Formula (I), and the intermediates used in the preparation thereof.

Another embodiment is a product obtainable by any of the processes or examples disclosed herein.

### Medical and Pharmaceutical Use

The compounds of Formula (I) and their pharmaceutically acceptable salts are believed to be useful in the prevention or treatment of cardiovascular disease and metabolic conditions, including but not limited to type 2 diabetes (T2D), obesity, non-alcoholic fatty liver disease (NAFLD) and non-alcoholic steatohepatitis (NASH), in a mammal, particularly a human.

For the avoidance of doubt, as used herein, the term “treatment” includes therapeutic and/or prophylactic treatment.

When a compound or salt described herein is administered as therapy for treating a disorder, a “therapeutically effective amount” is an amount sufficient to reduce or completely alleviate symptoms or other detrimental effects of the disorder, cure the disorder, reverse, completely stop, or slow the progress of the disorder or reduce the risk of the disorder getting worse.

The compounds described herein are thus indicated both in the therapeutic and/or prophylactic treatment of these conditions.

The compounds described herein have the advantage that they may be more efficacious, be less toxic, be more selective, be more potent, produce fewer side effects, be more easily absorbed, and/or have a better pharmacokinetic profile (e.g. higher oral bioavailability and/or lower clearance), than compounds known in the prior art.

For the above-mentioned therapeutic indications, the dosage administered will vary with the compound employed, the mode of administration and the treatment desired. However, in general, satisfactory results are obtained when the compounds are administered at a dosage of the solid form of between 1 mg and 2000 mg per day.

The compounds of Formula (I), and pharmaceutically acceptable derivatives thereof, may be used on their own, or in the form of appropriate pharmaceutical compositions in which the compound or derivative is in admixture with a pharmaceutically acceptable adjuvant, diluent or carrier. Thus, another aspect concerns a pharmaceutical composition comprising a novel compound of Formula (I), or a pharmaceutically acceptable salt thereof, in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical Formulations are described in, for example, Pharmaceuticals—The Science of Dosage Form Designs, M. E. Aulton, Churchill Livingstone, 2^{nd} Ed. 2002. In one embodiment the pharmaceutical composition preferably comprises less than 80% and in another embodiment less than 50% of a compound of Formula (I), or a pharmaceutically acceptable salt thereof.

In one embodiment there is provided a compound selected from any one of the compounds of Formula (I), or a pharmaceutically acceptable salt of a compound of Formula (I), for use in therapy, especially in the prevention or treatment of cardiovascular disease and metabolic conditions, including but not limited to type 2 diabetes (T2D), obesity, non-alcoholic fatty liver disease (NAFLD) and non-alcoholic steatohepatitis (NASH).

These and other embodiments are described in greater detail herein below, where further aspects will be apparent to one skilled in the art from reading this specification.

### Combination Therapy

The compounds of Formula (I), or pharmaceutically acceptable salts thereof, may also be administered in conjunction with other compounds used for the treatment of the above conditions.

In another embodiment, there is a combination therapy wherein a compound selected from any one of the compounds of Formula (I), or a pharmaceutically acceptable salt thereof, and a second active ingredient are administered concurrently, sequentially or in admixture, for the treatment of one or more of the conditions listed above. Such a combination may be used in combination with one or more further active ingredients.

When used in a combination therapy, it is contemplated that a compound selected from any one of the compounds of Formula (I), or pharmaceutically acceptable salts thereof, and the other active ingredients may be administered in a single composition, completely separate compositions, or a combination thereof. It also is contemplated that the active ingredients may be administered concurrently, simultaneously, sequentially, or separately. The particular composition(s) and dosing frequency(ies) of the combination therapy will depend on a variety of factors, including, for example, the route of administration, the condition being treated, the species of the patient, any potential interactions between the active ingredients when combined into a single composition, any interactions between the active ingredients when they are administered to the animal patient, and various other factors known to physicians (in the context of human patients), veterinarians (in the context of non-human patients), and others skilled in the art.

### Pharmaceutical Compositions

There is provided a method of treatment of a condition where modulation of GLP-1 receptor is required, which method comprises administration of a therapeutically effective amount of a compound selected from any one of the compounds of Formula (I) to a person suffering from, or susceptible to, such a condition.

The compounds of Formula (I) will normally be administered via the oral, topical, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable salt thereof, in a pharmaceutically acceptable dosage form. Depending upon the disorder and patient to be treated and the route of administration, the compositions may be administered at varying doses. Conventional procedures for the selection and preparation of suitable pharmaceutical Formulations are described in, for example, Pharmaceuticals—The Science of Dosage Form Designs, M. E. Aulton, Churchill Livingstone, 2^{nd} Ed. 2002.

In one embodiment suitable daily doses of the compounds of Formula (I) in therapeutical treatment of humans are about 0.0001-100 mg/kg body weight, in another embodiment 0.01-10 mg/kg body weight.

The optimum dosage and frequency of administration will depend on the particular condition being treated and its severity; the species of the patient; the age, sex, size and weight, diet, and general physical condition of the particular patient; brain/body weight ratio; other medication the patient may be taking; the route of administration; the Formulation; and various other factors known to physicians and others skilled in the art.

According to a further aspect there is thus provided a pharmaceutical Formulation comprising a compound selected from any one of the compounds of Formula (I), or pharmaceutically acceptable derivatives thereof, in admixture with a pharmaceutically acceptable adjuvant, diluent and/or carrier.

The compound of Formula (I) may be present in the pharmaceutical Formulation in a concentration from 0.1 to 99.5%, such as from 0.5 to 95%, by weight of the total Formulation.

### Preparation of the Compounds

The protection and deprotection of functional groups is described in Protective Groups in Organic Synthesis, 4^{th} Ed, T. W. Greene and P. G. M. Wuts, Wiley-Interscience (2006) and Protecting Groups, 3^{rd} Ed, P. J. Kocienski, Georg Thieme Verlag (2005).

A further embodiment encompasses pharmaceutically acceptable salts of the compounds of Formula (I).

A salt of a compound selected from any one of Formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound. In some embodiments (particularly where the salt is intended for administration to an animal, e.g. a human, or is a reagent for use in making a compound or salt intended for administration to an animal), the salt is pharmaceutically acceptable.

The term “pharmaceutically acceptable” is used to characterize a moiety (e.g. a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

Where the compound is sufficiently basic, pharmaceutically acceptable salts include, but are not limited to, inorganic or organic acid addition salts.

For reviews on suitable salts, see Berge et al., J. Pharm. Sci., 1977, 66, 1-19 or Handbook of Pharmaceutical Salts: Properties, selection and use, P. H. Stahl, P. G. Vermuth, IUPAC, Wiley-VCH, 2002.

Where an acid co-former is a solid at r.t. and there is no or only partial proton transfer between the compound of Formula (I) and such an acid co-former, a co-crystal of the co-former and compound of Formula (I) may result rather than a salt. All such co-crystal forms of the compound of Formula (I) are encompassed herein.

It is also to be understood that certain compounds of Formula (I) may exist in solvated form, e.g. hydrates, including solvates of a pharmaceutically acceptable salt of a compound of Formula (I).

In a further embodiment, certain compounds of Formula (I) may exist as racemates and racemic mixtures, single enantiomers, individual diastereomers and diastereomeric mixtures. Certain compounds of Formula (I) may also contain linkages (e.g. carbon-carbon bonds, carbon-nitrogen bonds such as amide bonds) wherein bond rotation is restricted about that particular linkage, e.g. restriction resulting from the presence of a ring bond or double bond. Stereoisomers may be separated using conventional techniques, e.g. chromatography or fractional crystallization, or the stereoisomers may be made by stereoselective synthesis.

In a further embodiment, the compounds of Formula (I) encompass any isotopically-labelled (or “radio-labelled”) derivatives of a compound of Formula (I). Such a derivative is a derivative of a compound of Formula (I) wherein one or more atoms are replaced by an atom having an atomic mass or mass number different from the atomic mass or mass number typically found in nature. Examples of isotopes that may be incorporated include ²H (also written as “D” for deuterium).

In a further embodiment, the compounds of Formula (I) may be administered in the form of a prodrug which is broken down in the human or animal body to give a compound of the Formula (I).

Various forms of prodrugs are known in the art. For examples of prodrug derivatives, see: Nature Reviews Drug Discovery 2008, 7, 255 and references cited therein. Intermediate compounds may also exist in enantiomeric forms and may be used as purified enantiomers, diastereomers, racemates or mixtures.

### EXAMPLES

The following examples are non-limiting examples.

### General Conditions

(i) operations were carried out at room temperature (rt), i.e. in the range 17 to 25□C and under an atmosphere of an inert gas such as N₂ unless otherwise stated;
(ii) where reactions refer to being degassed or purged, this can be performed for example by purging the reaction solvent with a constant flow of nitrogen for a suitable period of time (for example 5 to 10 min) or by repeatedly evacuating the vessel and backfill with appropriate inert atmosphere (for example nitrogen (g) or argon (g));
(iii) where reactions refer to the use of a microwave reactor, one of the following microwave reactors were used: Biotage Initiator, Personal Chemistry Emrys Optimizer, Personal Chemistry Smith Creator or CEM Explorer;
(iv) in general, the course of reactions was followed by thin layer chromatography (TLC) and/or analytical high performance liquid chromatography (HPLC or UPLC) which was usually coupled to a mass spectrometer (LCMS).
(v) when necessary, organic solutions were dried over anhydrous MgSO₄ or Na₂SO₄, or by using ISOLUTE® Phase Separator, and work-up procedures were carried out using traditional phase separating techniques.
(vi), evaporations were carried out either by rotary evaporation in vacuo or in a Genevac HT-4/EZ-2 or Biotage V10;
(vii) unless otherwise stated, flash column chromatography was performed on straight phase silica, using either Merck Silica Gel (Art. 9385) or prep-packed cartridges such as Biotage® SNAP cartridges (40-63 m silica, 4-330 g), Biotage® Sfar Silica HC D cartridges (20 μm, 10-100 g), Interchim puriFlash™ cartridges (25 μm, 4-120 g), Interchim puriFlash™ cartridges (50 μm, 25-330 g), Grace™ GraceResolv™ Silica Flash Cartridges (4-120 g) or Agela Flash Colum Silica-CS cartridges (80-330 g), or on reversed phase silica using Agela Technologies C-18, spherical cartridges (20-35 μm, 100 A, 80-330 g), manually or automated using a Grace Reveleris® X2 Flash system or similar system;
(viii) preparative reverse phase HPLC and preparative reverse phase SFC were performed using standard HPLC and SFC instruments, respectively, equipped with either a MS and/or UV triggered fraction collecting instrument, using either isocratic or a gradient of the mobile phase as described in the experimental section, and one of the following methods as described below;

### HPLC Prep Methods:

PrepMethod A: The compound was purified by preparative HPLC on a Waters™ Sunfire™ C18 OBD column (5 μm, 100×19 mm ID) using a gradient of MeCN in H₂O as mobile phase.
PrepMethod B: The compound was purified by preparative HPLC on a Chromatorex C18 SMB100-5T column (5 μm, 100×19 mm ID) using a gradient of MeCN in H₂O as mobile phase.
PrepMethod C: The compound was purified by preparative HPLC on a Kromasil C8 column (10 μm, 250×50 mm ID) using a gradient of MeCN in H₂O/MeCN/FA (95/5/0.2) as mobile phase.
PrepMethod D: The compound was purified by preparative HPLC on a Waters™ Sunfire™ C18 column (5 m, 150×30 mm ID) using a gradient of MeCN in H₂O/FA (0.05%) as mobile phase.
PrepMethod E: The compound was purified by preparative HPLC on an XBridge Shield RP18 OBD column (10 μm, 250×19 mm ID) using a gradient of MeCN in a H₂O/NH₄HCO₃ (10 mM)/NH₃ (0.1%, aq) buffer system as mobile phase
PrepMethod F: The compound was purified by preparative HPLC on an YMC Triart C18 column (5 μm, 100×20 mm ID) using a gradient of MeCN in H₂O/NH₄OH (0.1%) as mobile phase.
PrepMethod G. The compound was purified by preparative HPLC on a XSelect CSH OBD column (5 μm, 150×30 mm ID) using a gradient of MeCN in H₂O/FA (0.1%) as mobile phase.
PrepMethod H: The compound was purified by preparative HPLC on an Welch Uitimate XB—C18 column (10 μm, 250×50 mm ID) using a gradient of MeCN in a H₂O/NH₄HCO₃ (10 mM) buffer system as mobile phase.

Relevant fractions were collected, combined and freeze-dried to give the purified compound or relevant fractions were collected, combined and concentrated at reduced pressure, extracted with DCM or EtOAc, and the organic phase was dried either over Na₂SO₄ or by using a phase-separator, and then concentrated at reduced pressure to give the purified compound.
(ix) chiral preparative chromatography was carried out using HPLC or SFC on a standard HPLC or SFC instruments, respectively, and using either isocratic or gradient run with mobile phase as described in the experimental section;
(x) yields, where present, are not necessarily the maximum attainable, and when necessary, reactions were repeated if a larger amount of the reaction product was required;
(xi) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;
(xii) in general, the structures of the end-products of the Formula (I) were confirmed by nuclear magnetic resonance (NMR) and/or mass spectral techniques; proton NMR chemical shift values were measured on the delta scale using Bruker Avance III 300, 400, 500 and 600 spectrometers, operating at ¹H frequencies of 300, 400, 500 and 600 MHz, respectively. The experiments were typically recorded at 25° C. Chemical shifts are given in ppm with the solvent as internal standard. Protons on heteroatoms such as NH and OH protons are only reported when detected in NMR and can therefore be missing. In certain instances, protons can be masked or partially masked by solvent peaks and will therefore either be missing and not reported or reported as multiplets overlapping with solvent. The following abbreviations have been used (and derivatives thereof, e.g. dd, doublet of doublets, ddd, doublet of doublet of doublets, dt, doublet of triplets, dq, doublet of quartet etc.): s, singlet; d, doublet; t, triplet; q, quartet; m, multiplet; br, broad; qn, quintet; p, pentet; h, heptet. In some cases, the structures of the end-products of the Formula (I) might appear as rotamers in the NMR-spectrum, in which instances only peaks of the major rotamer are reported. Electrospray mass spectral data were obtained using a Waters Acquity UPLC coupled to a Waters single quadrupole mass spectrometer or similar equipment, acquiring both positive and negative ion data, and generally, only ions relating to the parent structure are reported; high resolution electrospray mass spectral data were obtained using a Waters XEVO qToF mass spectrometer or similar equipment, coupled to a Waters Acquity UPLC, acquiring either positive and negative ion data, and generally, only ions relating to the parent structure are reported
(xiii) intermediates were not necessarily fully purified but their structures and purity were assessed by TLC, analytical HPLC/UPLC, and/or NMR analysis and/or mass spectrometry;
(xiv) unless stated otherwise compounds containing an asymmetric carbon and/or sulfur atom were not resolved;
is (xv) in general Examples and Intermediate compounds are named using ChemDraw Professional version 20.1.1 from PerkinElmer. ChemDraw Professional version 20.1.1 generates the names of chemical structures using the Cahn-Ingold-Prelog (CIP) rules for stereochemistry and follows IUPAC rules as closely as possible when generating chemical names. Stereoisomers are differentiated from each other by stereodescriptors cited in names and assigned in accordance with the CIP rules.

ChemDraw is optionally using labels in the graphical representation of stereocenters such as ‘&’ and ‘or’ to describe the configuration of the stereochemical centers present in the structure.

In general chemical structures of Examples and Intermediates containing the label ‘&’ at a stereocenter, means the configuration of such Example or Intermediate at that stereocenter is a mixture of both (R) and (S); and a label ‘or’ means the configuration of such Example or Intermediate at that stereocenter is either (S) or (R). Absolute, unspecified, ‘&’, and ‘or’ stereocenters can all be present in a single structure.

In general for structures of Examples and Intermediates where all of the stereocenters are designated as ‘&’, the structure is named with a “rac-” prefix. For structures of Examples and Intermediates where all of the stereocenters are designated as ‘or’, the structure is named with a “rel-” prefix.

In general Examples and Intermediate compounds are named using the descriptors (RS) and (SR) to denote general ‘&’ centers for chemical structures with multiple chiral centers where only some are designated as ‘&’. The descriptors (R*) and (S*) are used to denote the general ‘or’ centers for chemical structures with multiple chiral centers where only some are designated as ‘or’.

In general Examples and Intermediate compounds containing stereocenters having a relationship that is either cis or trans, are named using the descriptors (RS, SR) or (RS, RS), to denote chemical structures with multiple chiral centers where only some are designated as ‘&’.

In general the descriptors (r) and (s) are used to describe the absolute configuration of any pseudoasymmetric centers in the structures of Examples and Intermediates.

In general the label “Isomer 1” corresponds to the first eluted isomer, and “Isomer 2” corresponds to the second eluted isomer, on a given chiral HPLC column and eluent, and are used to distinguish two isomers containing one or more stereocenters with absolute unknown configuration;
(xvi) in addition to the ones mentioned above, the following abbreviations and units have been used:

### Abbreviations

aq aqueous
calcd calculated
DCM dichloromethane
DIPEA N,N-diisopropylethylamine
DME dimethoxyethane
DMF N,N-dimethylformamide
DMSO dimethyl sulfoxide
Equiv equivalent
ESI electrospray ionization
EtOAc ethyl acetate
FA formic acid
(g) gas
HPLC high performance liquid chromatography
HRMS high resolution mass spectrometry
ID inner diameter
(l) liquid
LCMS liquid chromatography mass spectrometry
MeCN acetonitrile
MeOH methanol
MS mass spectrometry
NMR nuclear magnetic resonance
Pd(dppf)Cl₂·DCM [1,1′-Bis(diphenylphosphino)ferrocene]dichloropalladium(II) CH₂Cl₂ (1:1)
pTsOH para-toluenesulfonic acid
rt room temperature
sat saturated
SFC supercritical fluid chromatography
TFA trifluoroacetic acid
is THE tetrahydrofuran
TLC thin layer chromatography
UPLC ultra performance liquid chromatography
UV ultraviolet

### Units

atm atmosphere
C Celcius
g gram
h hour(s)
M mole per liter
mg milligram
MHz megaherz
min minute(s)
mL milliliter
mm millimeter
mmol millimole(s)
μCi microcurie
μL microliter
μmol micromole(s)
ppm parts per million
W/v weight by volume

### INTERMEDIATES

### Intermediate 1

### 4-(((6-Bromo-5-fluoropyridin-2-yl)oxy)methyl)-3-fluorobenzonitrile

3-Fluoro-4-(hydroxymethyl)benzonitrile (1.71 g, 11.34 mmol) and K₂CO₃ (3.13 g, 22.68 mmol) were added to a solution of 2-bromo-3,6-difluoropyridine (2.2 g, 11.34 mmol) in DMF (22 mL) at rt, and the reaction mixture was stirred at 90° C. for 14 h. The reaction mixture was cooled to rt, water (200 mL) was added, and the mixture was extracted with EtOAc (2×150 mL). The combined organic layer was washed with brine (100 mL), dried over Na₂SO₄ and concentrated at reduced pressure to give the title compound (3.9 g, 98%); ¹H NMR (500 MHz, CDCl₃) δ 7.64 (td, 1H), 7.48 (d, 1H), 7.44-7.36 (m, 2H), 6.76 (dq, 1H), 5.45 (s, 2H).

### Intermediate 2

### tert-Butyl (1R,5S,6r)-6-(6-((4-cyano-2-fluorobenzyl)oxy)-3-fluoropyridin-2-yl)-3-azabicyclo[3.1.0]hexane-3-carboxylate

Pd(dppf)Cl₂·DCM (75 mg, 92 μmol) and Na₂CO₃ (195 mg, 1.84 mmol) were added to a stirred solution of 4-[(6-bromo-5-fluoropyridin-2-yl)oxy]methyl-3-fluorobenzonitrile Intermediate 1 (300 mg, 923 μmol) and tert-butyl (1R,5S,6s)-6-(4,4,5,5-tetramethyl-1,3,2-dioxaborolan-2-yl)-3-azabicyclo[3.1.0]hexane-3-carboxylate (285 mg, 922 μmol) in DME:H₂O (2:1, 12 mL) under an atmosphere of argon (g) at 15° C. The mixture was degassed and refilled with argon (g) (×3). The reaction mixture was heated to 80° C. under an atmosphere of argon (g) for 18 h. The reaction mixture was cooled to rt. EtOAc (15 mL) and water (10 mL) was added and the mixture was filtered. The layers were separated and the aqueous layer was extracted with EtOAc (15 mL). The combined organic layer was dried over Na₂SO₄, filtered and concentrated under reduced pressure to give the crude title compound (1.2 g); MS (ESI) m/z [M+H]⁺ 428.2.

### Intermediate 3

### 4-(((6-((1R,5S,6r)-3-Azabicyclo[3.1.0]hexan-6-yl)-5-fluoropyridin-2-yl)oxy)methyl)-3-fluorobenzonitrile

TFA (1.48 g, 12.97 mmol) was added to a solution of tert-butyl (1R,5S,6r)-6-(6-((4-cyano-2-fluorobenzyl)oxy)-3-fluoropyridin-2-yl)-3-azabicyclo[3.1.0]hexane-3-carboxylate Intermediate 2 (370 mg, 866 μmol) in DCM (21 mL). The solution was stirred at ambient temperature for 8 h and then concentrated at reduced pressure to give the crude TFA salt of the title compound (800 mg).

### Intermediate 4

### Methyl (S)-3-methoxy-4-nitro-5-((oxetan-2-ylmethyl)amino)benzoate

K₂CO₃ (5.43 g, 39.27 mmol) was added to a solution of methyl 3-fluoro-5-methoxy-4-nitrobenzoate (3 g, 13.09 mmol) and (S)-oxetan-2-ylmethanamine (1.14 g, 13.09 mmol) in THF:DMF (5:2, 110 mL) and the reaction mixture was stirred at 90° C. for 16 h. The solvent was removed under reduced pressure, and the residue was suspended in water (250 mL). The aqueous layer was extracted with EtOAc (3×250 mL), and the combined organic layer was dried over Na₂SO₄, filtered and evaporated at reduced pressure. The crude product was purified by flash column chromatography on silica (10-20% EtOAc in petroleum ether) to give the title compound (1.8 g, 46%) as a yellow solid; MS (ESI) m/z [M+H]⁺ 297.1.

### Intermediate 5

### Methyl (S)-4-amino-3-methoxy-5-((oxetan-2-ylmethyl)amino)benzoate

A suspension of Pd—C (0.144 g, 1.35 mmol) and methyl (S)-3-methoxy-4-nitro-5-((oxetan-2-ylmethyl)amino)benzoate Intermediate 4 (4 g, 13.50 mmol) in THE (100 mL) was stirred under an atmosphere of H₂ (g) at 2 atm and 15° C. for 3 h. The reaction mixture was filtered through Celite® and the filter cake was washed with MeOH (3×300 mL). The filtrate was concentrated under reduced pressure. The crude product was purified by flash column chromatography on silica (50-70% EtOAc in petroleum ether) to give the title compound (3.00 g, 83%) as a light yellow solid; MS (ESI) m/z [M+H]⁺ 267.3.

### Intermediate 6

### Methyl (S)-2-(chloromethyl)-4-methoxy-1-(oxetan-2-ylmethyl)-1H-benzo[d]imidazole-6-carboxylate

pTsOH (0.119 g, 0.63 mmol) was added to a solution of methyl (S)-4-amino-3-methoxy-5-((oxetan-2-ylmethyl)amino)benzoate Intermediate 5 (0.333 g, 1.25 mmol) and 2-chloro-1,1,1-trimethoxyethane (0.387 g, 2.50 mmol) in MeCN (10 mL) and the reaction mixture was stirred at 45° C. for 30 min. The reaction mixture was concentrated under reduced pressure and the residue was purified by flash column chromatography on silica (50-100% EtOAc in heptane) to give the title compound (0.155 g, 38%); MS (ESI) m/z [M+H]⁺ 325.0.

### Intermediate 7

### Methyl 2-(((1R,5S,6S)-6-(6-((4-cyano-2-fluorobenzyl)oxy)-3-fluoropyridin-2-yl)-3-azabicyclo[3.1.0]hexan-3-yl)methyl)-4-methoxy-1-(((S)-oxetan-2-yl)methyl)-1H-benzo[d]imidazole-6-carboxylate

Methyl (S)-2-(chloromethyl)-4-methoxy-1-(oxetan-2-ylmethyl)-1H-benzo[d]imidazole-6-carboxylate Intermediate 6 (491 mg, 1.51 mmol) and DIPEA (521 mg, 4.03 mmol) were added to a solution of crude TFA salt of 4-(((6-((1R,5S,6r)-3-azabicyclo[3.1.0]hexan-6-yl)-5-fluoropyridin-2-yl)oxy)methyl)-3-fluorobenzonitrile Intermediate 3 (330 mg, 1.01 mmol) in DMF (5 mL) and the reaction mixture was stirred at rt for 14 h. The reaction mixture was concentrated at reduced pressure and the crude product was purified by preparative HPLC, PrepMethod A, (gradient: 50-75%) to give the title compound (241 mg, 32%); MS (ESI) m z [M+H]⁺ 616.0.

### Intermediate 8

### Methyl 2-(((1R,5S,6S)-6-(6-((4-cyano-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylate

K₂CO₃ (0.190 g, 1.37 mmol) was added in one portion to a solution of 4-(((6-((1R,5S,6r)-3-azabicyclo[3.1.0]hexan-6-yl)-5-fluoropyridin-2-yl)oxy)methyl)-3-fluorobenzonitrile Intermediate 3 (0.15 g, 0.46 mmol) and methyl (S)-2-(chloromethyl)-1-(oxetan-2-ylmethyl)-1H-benzo[d]imidazole-6-carboxylate (0.135 g, 0.46 mmol) in MeCN (5 mL), and the reaction mixture was stirred at 60° C. for 4 h. The reaction mixture was filtered through celite, and the filter cake was washed with MeCN (50 mL). The filtrate was collected and concentrated under reduced pressure. The residue was diluted with EtOAc (100 mL) and washed with sat brine (2×50 mL). The organic layer was dried over Na₂SO₄, filtered and evaporated at reduced pressure and the crude product was purified by preparative TLC (EtOAc:petroleum ether, 1:1) to give the title compound (0.120 g, 45%) as a yellow solid; MS (ESI) m/z [M+H]⁺ 586.

### Intermediate 9

### tert-Butyl (1R,5S,6r)-6-(6-((4-cyano-2-fluorobenzyl)oxy)pyridin-2-yl)-3-azabicyclo[3.1.0]hexane-3-carboxylate

Pd(dppf)Cl₂·DCM (82 mg, 0.10 mmol) was added in one portion to a mixture of 4-(((6-bromopyridin-2-yl)oxy)methyl)-3-fluorobenzonitrile (307 mg, 1.00 mmol), tert-butyl (1R,5S,6r)-6-(4,4,5,5-tetramethyl-1,3,2-dioxaborolan-2-yl)-3-azabicyclo[3.1.0]hexane-3-carboxylate (309 mg, 1.00 mmol) and K₂CO₃ (276 mg, 2.00 mmol) in 1,4-dioxane (5 mL) and water (1.67 mL) under an atmosphere of N₂ (g), and the reaction mixture was stirred at 80° C. for 3 h. The reaction mixture was filtered through Celite® and the filtrate was collected and concentrated under reduced pressure. The crude residue was purified by preparative TLC (EtOAc:petroleum ether, 1:2) to give the title compound (0.25 g, 61%) as a yellow solid; MS (ESI) m/z [M+H]⁺ 410.

### Intermediate 10

### 4-(((6-((1R,5S,6r)-3-Azabicyclo[3.1.0]hexan-6-yl)pyridin-2-yl)oxy)methyl)-3-fluorobenzonitrile

pTSOH (263 mg, 1.53 mmol) was added to a solution of tert-butyl (1R,5S,6r)-6-(6-((4-cyano-2-fluorobenzyl)oxy)pyridin-2-yl)-3-azabicyclo[3.1.0]hexane-3-carboxylate Intermediate 9 (250 mg, 0.61 mmol) in EtOAc (5 mL) at 25° C., and the reaction mixture was stirred at 60° C. for 3 h The precipitate was collected by filtration, washed with EtOAc and dried to give the para toluensulfonate salt of the title compound (0.35 g, 88%) as a white solid; MS (ESI) m z [M+H]⁺ 310.

### Intermediate 11

### Methyl 2-(((1R,5S,6S)-6-(6-((4-cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylate

pTsOH (5.09 mg, 0.03 mmol) was added to a solution of 2-chloro-1,1,1-trimethoxyethane (91 mg, 0.59 mmol) and methyl (S)-4-amino-3-((oxetan-2-ylmethyl)amino)benzoate (126 mg, 0.54 mmol) in MeCN (3 mL) at 20° C. under an atmosphere of N₂ (g), and the reaction mixture was stirred at 20° C. for 2 h. K₂CO₃ (296 mg, 2.14 mmol) and 4-(((6-((1R,5S,6r)-3-azabicyclo[3.1.0]hexan-6-yl)pyridin-2-yl)oxy)methyl)-3-fluorobenzonitrile para toluensulfonate Intermediate 10 (350 mg, 0.54 mmol) were added slowly to the mixture at 50° C., and under an atmosphere of N₂ (g). The resulting suspension was stirred at 50° C. for 2 h. The reaction mixture was diluted with EtOAc (100 mL) and washed sequentially with sat NaHCO₃ (10 mL), sat NH₄Cl (25 mL), and water (75 mL). The organic layer was dried over Na₂SO₄, filtered and concentrated at reduced pressure to afford the crude product. The residue was purified by preparative TLC (EtOAc:petroleum ether, 1:1), to give the title compound (0.30 g, 99%) as a white solid; MS (ESI) m/z [M+H]⁺ 568.

### Intermediate 12

### Methyl 2-(((1R,5S,6S)-6-(6-((4-cyano-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylate

4-(((6-((1R,5S,6r)-3-Azabicyclo[3.1.0]hexan-6-yl)pyridin-2-yl)oxy)methyl)-3-fluorobenzonitrile Intermediate 10 (150 mg, 485 μmol) and DIPEA (251 mg, 1.94 mmol) was added to a solution of methyl (S)-2-(chloromethyl)-4-methoxy-1-(oxetan-2-ylmethyl)-1H-benzo[d]imidazole-6-carboxylate Intermediate 6 (158 mg, 485 μmol) in MeCN (3 mL) at rt and the reaction mixture was stirred at rt for 14 h. The reaction mixture was concentrated at reduced pressure and the crude product was purified by preparative HPLC, PrepMethod A, (isocratic run: 60%), to give the title compound (135 mg, 47%); MS (ESI) m/z [M+H]⁺ 598.2.

### Intermediate 13

### Methyl 2-(((1R,5S,6S)-6-(6-((4-cyano-2-fluorobenzyl)oxy)pyridin-2-yl)-3-azabicyclo[3.1.0]hexan-3-yl)methyl)-5-fluoro-1-(((S)-oxetan-2-yl)methyl)-1H-benzo[d]imidazole-6-carboxylate

Methyl (S)-2-(chloromethyl)-5-fluoro-1-(oxetan-2-ylmethyl)-1H-benzo[d]imidazole-6-carboxylate WO2020103815 (525 mg, 1.68 mmol), 4-(((6-((1R,5S,6r)-3-azabicyclo[3.1.0]hexan-6-yl)pyridin-2-yl)oxy)methyl)-3-fluorobenzonitrile Intermediate 10 (520 mg, 1.68 mmol), DIPEA (730 μL, 543 mg, 4.2 mmol) and NaI (13 mg, 84 μmol) were mixed in MeCN (10 mL), and the reaction mixture was stirred at rt for 16 h. The reaction mixture was diluted with water and extracted with EtOAc. The organic layer was dried over Na₂SO₄, filtered and evaporated at reduced pressure. The residue was purified by preparative HPLC, PrepMethod B, gradient: 55-80%) to give the title compound (300 mg, 29%); MS (ESI) m/z [M+H]⁺ 586.2.

### Intermediate 14

### Methyl (S)-4-nitro-3-(((tetrahydrofuran-2-yl)methyl)amino)benzoate

DIPEA (6.91 mL, 39.55 mmol) was added to a solution of (S)-(tetrahydrofuran-2-yl)methanamine (2.0 g, 19.8 mmol) and methyl 3-fluoro-4-nitrobenzoate (3.94 g, 19.8 mmol) in MeCN (20 mL), and the reaction mixture was stirred at 35° C. for 18 h. The reaction mixture was evaporated and the residue was purified by flash chromatography on silica (0-40% EtOAc in heptane) to give the title compound (4.34 g, 78%); ¹H NMR (500 MHz, CDCl₃) 1.65-1.76 (1H, m), 1.94-2.03 (2H, m), 2.11 (1H, td), 3.35-3.43 (1H, m), 3.52 (1H, d), 3.79-3.86 (1H, m), 3.94 (4H, s), 4.22 (1H, qd), 7.24 (1H, dd), 7.59 (1H, d), 8.22 (2H, d).

### Intermediate 15

### Methyl (S)-4-amino-3-(((tetrahydrofuran-2-yl)methyl)amino)benzoate

Pd—C (5%, 0.33 g, 3.10 mmol) was added to a solution of methyl (S)-4-nitro-3-(((tetrahydrofuran-2-yl)methyl)amino)benzoate Intermediate 14 (4.34 g, 15.48 mmol) in MeOH (40 mL) and the reaction mixture was stirred under an atmosphere of H₂ (g) (1 atm) at rt for 30 min. The catalyst was filtered off and the filtrate was concentrated under reduced pressure to give the title compound (3.70 g, 95%); MS (ESI) m/z [M+H]⁺ 251.25.

### Intermediate 16

### Methyl (S)-2-(chloromethyl)-1-((tetrahydrofuran-2-yl)methyl)-1H-benzo[d]imidazole-6-carboxylate

pTsOH (0.281 g, 1.48 mmol) was added to a solution of methyl (S)-4-amino-3-(((tetrahydrofuran-2-yl)methyl)amino)benzoate Intermediate 15 (3.7 g, 14.78 mmol) and 2-chloro-1,1,1-trimethoxyethane (2.74 g, 17.74 mmol) in MeCN (10 mL), and the reaction mixture was stirred at 45° C. for 90 min. The solvent was evaporated and the residue was purified by flash chromatography on silica (50-100% EtOAc in heptane) to give the title compound (4.30 g, 94%); MS (ESI) m/z [M+H]⁺ 309.0.

### Intermediate 17

### Methyl 2-(((1R,5S,6S)-6-(6-((4-cyano-2-fluorobenzyl)oxy)pyridin-2-yl)-3-azabicyclo[3.1.0]hexan-3-yl)methyl)-1-(((S)-tetrahydrofuran-2-yl)methyl)-1H-benzo[d]imidazole-6-carboxylate

A mixture of 4-(((6-((1R,5S,6r)-3-azabicyclo[3.1.0]hexan-6-yl)pyridin-2-yl)oxy)methyl)-3-fluorobenzonitrile Intermediate 10 (261 mg, 0.68 mmol), methyl (S)-2-(chloromethyl)-1-((tetrahydrofuran-2-yl)methyl)-1H-benzo[d]imidazole-6-carboxylate Intermediate 16 (253 mg, 0.82 mmol), and DIPEA (500 μL, 2.86 mmol) in MeCN (4 mL) was stirred at 40° C. for 3 h. The reaction mixture was concentrated at reduced pressure and the residue was purified by preparative HPLC, PrepMethod C, (gradient: 35-75%), to give the title compound (0.252 g, 64%) as a colourless oil; MS (ESI) m/z [M+H]⁺ 582.4.

### Intermediate 18

### Methyl 3-(((1-ethyl-1H-imidazol-5-yl)methyl)amino)-5-methoxy-4-nitrobenzoate

A solution of methyl 3-fluoro-5-methoxy-4-nitrobenzoate (1.4 g, 6.11 mmol) in DMF (5 mL) was slowly added dropwise to a solution (1-ethyl-1H-imidazol-5-yl)methanamine dihydrochloride (1.33 g, 6.72 mmol) and DIPEA (3.95 g, 30.54 mmol) in DMF (5 mL) and the reaction mixture was heated under stirring at 60° C. for 16 h. The reaction mixture was cooled to rt, diluted with water (30 mL), and the mixture was extracted with DCM (3×30 mL). The combined organic layer was washed with brine (50 mL) and water (50 mL), dried over Na₂SO₄, filtered, and concentrated in vacuo. The crude product was purified by straight phase flash chromatography on silica (10% EtOAc in hexane) to give the title compound (1.9 g, 93%) as a yellow solid; MS (ESI) m/z [M+H]⁺ 335.1.

### Intermediate 19

### Methyl 4-amino-3-(((1-ethyl-1H-imidazol-5-yl)methyl)amino)-5-methoxybenzoate

10% Pd/C (0.2 g) was added to a solution of methyl 3-(((1-ethyl-1H-imidazol-5-yl)methyl)amino)-5-methoxy-4-nitrobenzoate Intermediate 18 (1.9 g, 5.0 mmol) in MeOH (20 mL), and the reaction mixture was stirred under an atmosphere of H₂(g) (1 atm) at rt for 36 h. The reaction mixture was filtered through a pad of Celite, the filtrate was concentrated in vacuo, and the crude product was purified by straight phase flash chromatography on silica (0-95% MeOH in MTBE) to give the title compound (1.5 g, 87%) as a yellow solid; MS (ESI) m/z [M+H]⁺ 305.2.

### Intermediate 20

### Methyl 1-((1-ethyl-1H-imidazol-5-yl)methyl)-2-(hydroxymethyl)-4-methoxy-1H-benzo[d]imidazole-6-carboxylate

2,2,2-Triethoxyethan-1-ol (2.63 g, 14.78 mmol) and pTsOH (424 mg, 2.46 mmol) were added to a solution of methyl 4-amino-3-(((1-ethyl-1H-imidazol-5-yl)methyl)amino)-5-methoxybenzoate Intermediate 19 (1.5 g, 4.93 mmol) in MeCN (7 mL), and the reaction mixture was heated under stirring at 60° C. for 1 h. The reaction mixture was cooled to rt and concentrated in vacuo, and the residue was purified by straight phase flash chromatography on silica (0-95% MeOH in MTBE) to give the title compound (1.4 g, 75%); ¹H NMR (400 MHz, DMSO-d₆) δ 7.74 (s, 1H), 7.65 (s, 1H), 7.26 (s, 1H), 6.52 (s, 1H), 5.75 (s, 1H), 5.65 (s, 2H), 4.70 (d, 2H), 3.95 (d, 5H), 3.83 (s, 3H), 1.11 (t, 3H).

### Intermediate 21

### Methyl 2-(chloromethyl)-1-((1-ethyl-1H-imidazol-5-yl)methyl)-4-methoxy-1H-benzo[d]imidazole-6-carboxylate

A catalytic amount of DMF followed by SOCl₂ (2.42 g, 20.32 mmol) were added dropwise under vigorous stirring to a solution of methyl 1-((1-ethyl-1H-imidazol-5-yl)methyl)-2-(hydroxymethyl)-4-methoxy-1H-benzo[d]imidazole-6-carboxylate Intermediate 20 (1.4 g, 4.07 mmol) in DCM (20 mL), and the reaction mixture was stirred at rt for 1 h. The reaction solution was concentrated in vacuo and the obtained solid was dried in vacuo to give the dihydrochloride salt of the title compound (1.70 g, 96%) as a beige solid; MS (ESI) m z [M+H]⁺ 363.2.

### Intermediate 22

### Methyl 2-(((1R,5S,6r)-6-(6-((4-cyano-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylate

The title compound was prepared from the HCl-salt of 4-(((6-((1R,5S,6r)-3-azabicyclo[3.1.0]hexan-6-yl)-5-fluoropyridin-2-yl)oxy)methyl)-3-fluorobenzonitrile Intermediate 3 (0.7 g, 1.9 mmol) and methyl 2-(chloromethyl)-1-((1-ethyl-1H-imidazol-5-yl)methyl)-4-methoxy-1H-benzo[d]imidazole-6-carboxylate Intermediate 21 (0.698 g, 1.9 mmol) in analogy with the description for Intermediate 7 to give the title compound (24 mg, 2%); MS (ESI) m/z [M+H]⁺ 654.2.

### Intermediate 23

### Methyl 4-chloro-2-(chloromethyl)-1H-benzo[d]imidazole-6-carboxylate

2-Chloro-1,1,1-trimethoxyethane (131 mg, 849 μmol) and pTsOH hydrate (3 mg, 15.4 μmol) were added to a solution of methyl 3,4-diamino-5-chlorobenzoate (155 mg, 772 μmol) in MeCN (2 mL) and the reaction mixture was stirred at 60° C. for 1 h. The reaction mixture was cooled to rt and concentrated in vacuo. The residue was diluted with water (5 mL), neutralized using sat NaHCO₃ and the aqueous layer was extracted with EtOAc (3×5 mL). The combined organic layer was washed with brine (10 mL) and water (10 mL), dried over Na₂SO₄, filtered, and concentrated in vacuo to give the title compound (0.18 g, 95%). ¹H NMR (400 MHz, CDCl₃) δ 8.26 (s, 1H), 8.05 (s, 1H), 4.92 (s, 2H), 3.97 (t, 3H), 1.69 (br.s, 1H, NH exchange).

### Intermediate 24

### Methyl 4-chloro-2-(((1R,5S,6r)-6-(6-((4-cyano-2-fluorobenzyl)oxy)pyridin-2-yl)-3-azabicyclo[3.1.0]hexan-3-yl)methyl)-1H-benzo[d]imidazole-6-carboxylate

The title compound was prepared from 4-(((6-((1R,5S,6r)-3-azabicyclo[3.1.0]hexan-6-yl)pyridin-2-yl)oxy)methyl)-3-fluorobenzonitrile Intermediate 10 (378 mg, 1.22 mmol) and methyl 4-chloro-2-(chloromethyl)-1H-benzo[d]imidazole-6-carboxylate Intermediate 23 in analogy with the description for Intermediate 12 (317 mg, 1.22 mmol). The reaction mixture was concentrated in vacuo, diluted with water, and extracted with EtOAc (×3). The combined organic layer was washed with brine and water, dried over Na₂SO₄, filtered and concentrated in vacuo to give the title compound (530 mg, 81%); MS (ESI) m/z [M+H]⁺ 532.0.

### Intermediate 25

### Methyl 4-chloro-2-(((1R,5S,6S)-6-(6-((4-cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylate

(S)-2-(Iodomethyl)oxetane (197 mg, 997 μmol) was added to a suspension of methyl 4-chloro-2-(((1R,5S,6r)-6-(6-((4-cyano-2-fluorobenzyl)oxy)pyridin-2-yl)-3-azabicyclo[3.1.0]hexan-3-yl)methyl)-1H-benzo[d]imidazole-6-carboxylate Intermediate 24 (530 mg, 997 μmol) and CsCO₃ (487 mg, 1.49 mmol) in MeCN (5 mL) and the reaction mixture was stirred at 45° C. for 18 h. The reaction mixture was concentrated in vacuo, the residue was dissolved in water (10 mL) and the resulting mixture was extracted with EtOAc (3×10 mL). The combined organic layer was washed with brine and water, dried over Na₂SO₄, filtered, and concentrated in vacuo. The residue was purified by preparative HPLC, PrepMethod A (gradient: 0-65%), to give the title compound (358 mg, 60% yield); MS (ESI) m/z [M+H]⁺ 602.2.

### Intermediate 26

### Methyl 2-(chloromethyl)-4-methyl-1H-benzo[d]imidazole-6-carboxylate

The title compound was prepared from methyl 3,4-diamino-5-methylbenzoate (1.0 g, 5.55 mmol) in analogy with Intermediate 23. The reaction mixture was allowed to attain rt, filtered and dried in vacuo to give the title compound (1.2 g, 85%); MS (ESI) m/z [M+H]⁺ 239.0.

### Intermediate 27

### Methyl 2-(((1R,5S,6r)-6-(6-((4-cyano-2-fluorobenzyl)oxy)pyridin-2-yl)-3-azabicyclo[3.1.0]hexan-3-yl)methyl)-4-methyl-1H-benzo[d]imidazole-6-carboxylate

The title compound was prepared from 4-(((6-((1R,5S,6r)-3-azabicyclo[3.1.0]hexan-6-yl)pyridin-2-yl)oxy)methyl)-3-fluorobenzonitrile Intermediate 10 (594 mg, 2.49 mmol) and methyl 2-(chloromethyl)-4-methyl-1H-benzo[d]imidazole-6-carboxylate Intermediate 26 (700 mg, 2.26 mmol) in analogy with Intermediate 12 except that the reaction mixture was heated at 50° C. for 16 h. The reaction mixture was cooled, diluted with water, and extracted several times with EtOAc. The combined organic layer was washed with brine, dried over Na₂SO₄, filtered, and concentrated under reduced pressure. The crude residue was purified by preparative HPLC, PrepMethod A (gradient: 45-75%), to give the title compound (0.2 g, 37%); MS (ESI) m/z [M+H]⁺ 512.4.

### Intermediate 28

### Methyl 2-(((1R,5S,6S)-6-(6-((4-cyano-2-fluorobenzyl)oxy)pyridin-2-yl)-3-azabicyclo[3.1.0]hexan-3-yl)methyl)-4-methyl-1-(((S)-oxetan-2-yl)methyl)-1H-benzo[d]imidazole-6-carboxylate

The title compound was prepared from methyl 2-(((1R,5S,6r)-6-(6-((4-cyano-2-fluorobenzyl)oxy)pyridin-2-yl)-3-azabicyclo[3.1.0]hexan-3-yl)methyl)-4-methyl-1H-benzo[d]imidazole-6-carboxylate Intermediate 27 (220 mg, 430 μmol) in analogy with the description for Intermediate 25, but using DMF (1 mL) as solvent instead of MeCN, to give the title compound (200 mg, 72%); ¹H NMR (400 MHz, CDCl₃) δ 7.98 (s, 1H), 7.80 (s, 1H), 7.59 (t, 1H), 7.45 (d, 2H), 7.38-7.33 (m, 1H), 6.79 (s, 1H), 6.58 (d, 1H), 5.44 (s, 2H), 4.63 (q, 1H), 4.39 (dt, 1H), 3.95 (d, 3H), 3.23-3.13 (m, 1H), 2.69 (d, 3H), 2.62 (d, 2H), 2.45 (t, 1H), 2.02 (s, 2H), 1.95 (t, 1H), 1.37 (s, 4H), 1.27 (s, 3H).

### Intermediate 29

### Methyl 2-(chloromethyl)-1-((1-ethyl-1H-imidazol-5-yl)methyl)-1H-benzo[d]imidazole-6-carboxylate

The title compound was prepared in four steps from methyl 3-fluoro-4-nitrobenzoate (2.1 g, 10.55 mmol) in analogy with the description for Intermediate 21 to give the title compound (2.1 g, 95%); MS (ESI) m/z [M+H]⁺ 333.2.

### Intermediate 30

### Methyl 2-(((1R,5S,6r)-6-(6-((4-cyano-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylate

The title compound was prepared from 4-(((6-((1R,5S,6r)-3-azabicyclo[3.1.0]hexan-6-yl)pyridin-2-yl)oxy)methyl)-3-fluorobenzonitrile Intermediate 10 (400 mg, 1.29 mmol) and methyl 2-(chloromethyl)-1-((1-ethyl-1H-imidazol-5-yl)methyl)-1H-benzo[d]imidazole-6-carboxylate Intermediate 29 (430 mg, 1.29 mmol) in analogy with Intermediate 12, except that the reaction mixture was heated at 50° C. for 16 h. The reaction mixture was cooled, diluted with water, and extracted several times with EtOAc. The combined organic layer was washed with brine, dried over Na₂SO₄, filtered and evaporated. The crude residue was purified by preparative HPLC, PrepMethod A (gradient: 40-60%), to give the title compound: MS (ESI) m/z [M+H]⁺ 606.2.

### Intermediate 31

### tert-Butyl (1R,5S,6r)-6-(6-((4-chloro-2-fluorobenzyl)oxy)pyridin-2-yl)-3-azabicyclo[3.1.0]hexane-3-carboxylate

The title compound was prepared from 2-bromo-6-((4-chloro-2-fluorobenzyl)oxy)pyridine WO2020207474 (1.02 g, 3.22 mmol) and tert-butyl (1R,5S,6s)-6-(4,4,5,5-tetramethyl-1,3,2-dioxaborolan-2-yl)-3-azabicyclo[3.1.0]hexane-3-carboxylate (1.1 g, 3.55 mmol) in analogy with the description for Intermediate 2. The crude product was purified by straight phase chromatography on silica (hexane:MTBE, 1:1) to give the title compound (440 mg, 28%); MS (ESI) m/z [M+H]⁺ 419.0.

### Intermediate 32

### (1R,5S,6r)-6-(6-((4-Chloro-2-fluorobenzyl)oxy)pyridin-2-yl)-3-azabicyclo[3.1.0]hexane

tert-Butyl (1R,5S,6r)-6-(6-((4-chloro-2-fluorobenzyl)oxy)pyridin-2-yl)-3-azabicyclo[3.1.0]hexane-3-carboxylate Intermediate 31 (436 mg, 1.04 mmol) was dissolved in 4 M HCl in 1,4-dioxane (20 mL) and the reaction mixture was stirred at rt overnight. The precipitate was filtered off, washed with cold 1,4-dioxane (5 mL) and dried in vacuo to give the HCl salt of the title compound (170 mg, 46%); MS (ESI) m/z [M+H]⁺ 319.0.

### Step c)

### Intermediate 33

### (1R,5S,6r)-6-(6-((4-Chloro-2-fluorobenzyl)oxy)pyridin-2-yl)-3-azabicyclo[3.1.0]hexane

A solution of tert-butyl (1R,5S,6r)-6-(6-((4-chloro-2-fluorobenzyl)oxy)pyridin-2-yl)-3-azabicyclo[3.1.0]hexane-3-carboxylate Intermediate 31 (850 mg, 2.03 mmol) in DCM (0.6 mL) and TFA (0.2 mL) was stirred at 15° C. for 1 h. The reaction mixture was concentrated in vacuo to give the TFA salt of the title compound (1.3 g, 97%); MS (ESI) m/z [M+H]⁺ 319.0.

### Intermediate 34

### Methyl 2-(((1R,5S,6S)-6-(6-((4-chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylate

The title compound was prepared from (1R,5S,6r)-6-(6-((4-chloro-2-fluorobenzyl)oxy)pyridin-2-yl)-3-azabicyclo[3.1.0]hexane TFA Intermediate 33 (244 mg, 0.37 mmol) and methyl (S)-2-(chloromethyl)-4-methoxy-1-(oxetan-2-ylmethyl)-1H-benzo[d]imidazole-6-carboxylate Intermediate 6 (120 mg, 0.37 mmol) in analogy with the description for Intermediate 8. The crude product was purified by preparative TLC (DCM:MeOH, 10:1) to give the title compound (170 mg, 76%); MS (ESI) m/z [M+H]⁺ 607.

### Intermediate 35

### Methyl 2-(((1R,5S,6r)-6-(6-((4-chloro-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylate

The title compound was prepared from (1R,5S,6r)-6-(6-((4-chloro-2-fluorobenzyl)oxy)pyridin-2-yl)-3-azabicyclo[3.1.0]hexane HCl Intermediate 32 (249 mg, 0.782 mmol) and methyl 2-(chloromethyl)-1-((1-ethyl-1H-imidazol-5-yl)methyl)-1H-benzo[d]imidazole-6-carboxylate Intermediate 29 (400 mg, 1.2 mmol) in analogy with the description for Intermediate 12, except that the reaction mixture was heated at 50° C. for 16 h, to give the title compound (154 mg, 24%); MS (ESI) m/z [M+H]⁺ 615.2.

### Intermediate 36

### Methyl 2-(((1R,5S,6S)-6-(6-((4-chloro-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylate

The title compound was prepared from (1R,5S,6r)-6-(6-((4-chloro-2-fluorobenzyl)oxy)pyridin-2-yl)-3-azabicyclo[3.1.0]hexane HCl Intermediate 32 (169 mg, 0.477 mmol) and methyl (S)-2-(chloromethyl)-1-(oxetan-2-ylmethyl)-1H-benzo[d]imidazole-6-carboxylate (169 mg, 0.572 mmol) in analogy with the description for Intermediate 13 to give the title compound (275 mg, 92%); ¹H NMR (400 MHz, CDCl₃) δ 8.3 (s., 1H), 7.9 (d, 1H) 7.7 (d, 1H), 7.45-7.3 (m, 2H), 7.15-7.05 (m, 2H), 6.7 (d, 1H), 6.5 (d, 1H), 5.3 (s, 2H), 5.15 (m, 1H), 4.7-4.55 (m, 3H), 4.35 (m, 1H), 4.05 (s, 2H), 3.9 (s, 3H), 3.05 (t, 2H), 2.75-2.6 (m, 3H), 2.45 (m, 1H), 2.2 (s, 1H), 1.85 (s, 2H).

### Intermediate 37

### Methyl 2-(((1R,5S,6r)-6-(6-((4-chloro-2-fluorobenzyl)oxy)pyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylate

The title compound was prepared from (1R,5S,6r)-6-(6-((4-chloro-2-fluorobenzyl)oxy)pyridin-2-yl)-3-azabicyclo[3.1.0]hexane TFA Intermediate 33 (328 mg, 0.50 mmol) and methyl 2-(chloromethyl)-1-((1-ethyl-1H-imidazol-5-yl)methyl)-4-methoxy-1H-benzo[d]imidazole-6-carboxylate Intermediate 21 (120 mg, 0.33 mmol) in analogy with the description for Intermediate 17, except that the reaction mixture was heated at 80° C. for 16 h. The reaction mixture was concentrated in vacuo and the crude product was purified by preparative TLC (DCM:MeOH, 10:1) to give the title compound (140 mg, 66%) as a yellow solid; MS (ESI) m/z [M+H]⁺ 645.

### Intermediate 38

### Methyl (S)-3-fluoro-4-nitro-5-((oxetan-2-ylmethyl)amino)benzoate

DIPEA (11.90 g, 0.092 mol) was added to a solution of methyl 3,5-difluoro-4-nitrobenzoate (10 g, 0.046 mol) in THE (100 mL), and the reaction mixture was stirred for 10 min. A solution of (S)-oxetan-2-ylmethanamine (4.01 g, 0.046 mol) in THE (20 mL) was added, and the reaction mixture was stirred at 25° C. for 16 h. EtOAc (20 mL) was added to the reaction mixture, and the mixture was filtered through silica gel. The filter cake was rinsed with EtOAc and the combined filtrates were concentrated at reduced pressure at 45° C., to give the title compound (12 g, 92%); MS (ESI) m/z [M+H]⁺ 285.0.

### Intermediate 39

### Methyl (S)-4-amino-3-fluoro-5-((oxetan-2-ylmethyl)amino)benzoate

Methyl (S)-3-fluoro-4-nitro-5-((oxetan-2-ylmethyl)amino)benzoate Intermediate 38 (12 g, 0.042 mol) was dissolved in THE (100 mL) and treated with 10% Pd/C (1.2 g). The reaction mixture was stirred under an atmosphere of H₂ (g) at ambient pressure and temperature for 24 h. The reaction mixture was filtered, and the filtrate was concentrated at reduced pressure. The residue was purified by straight phase flash chromatography on silica (0-95% MeCN in CHCl₃) to give the title compound (8.5 g, 79%); MS (ESI) m/z [M+H]⁺ 255.0.

### Intermediate 40

### Methyl (S)-2-(chloromethyl)-4-fluoro-1-(oxetan-2-ylmethyl)-1H-benzo[d]imidazole-6-carboxylate

2-Chloro-1,1,1-trimethoxyethane (3.64 g, 0.023 mol, 3.18 ml) and pTSOH (127 mg, 669 μmol) was added to a solution of methyl (S)-4-amino-3-fluoro-5-((oxetan-2-ylmethyl)amino)benzoate Intermediate 39 (5 g, 0.019 mol) in THE (50 mL), and the reaction mixture was stirred at 50° C. overnight. The reaction mixture was poured into water and the mixture was extracted with EtOAc. The organic layer was washed with brine and concentrated in vacuo to give the title compound (6 g, 95%); MS (ESI) m/z [M+H]⁺ 285.0.

### Intermediate 41

### Methyl 2-(((1R,5S,6S)-6-(6-((4-chloro-2-fluorobenzyl)oxy)pyridin-2-yl)-3-azabicyclo[3.1.0]hexan-3-yl)methyl)-4-fluoro-1-(((S)-oxetan-2-yl)methyl)-1H-benzo[d]imidazole-6-carboxylate

The title compound was prepared from (1R,5S,6r)-6-(6-((4-chloro-2-fluorobenzyl)oxy)pyridin-2-yl)-3-azabicyclo[3.1.0]hexane TFA Intermediate 33 (254 mg, 0.38 mmol) and methyl (S)-2-(chloromethyl)-4-fluoro-1-(oxetan-2-ylmethyl)-1H-benzo[d]imidazole-6-carboxylate Intermediate 40 (120 mg, 0.38 mmol) in analogy with the description for Intermediate 8. The crude product was purified by preparative TLC (DCM:MeOH, 10:1) to give the title compound (160 mg, 70%); MS (ESI) m/z [M+H]⁺ 595.

### Intermediate 42

### 2-Bromo-6-((4-chloro-2-fluorobenzyl)oxy)-3-fluoropyridine

(4-Chloro-2-fluorophenyl)methanol (248 mg, 1.55 mmol) and K₂CO₃ (427 mg, 3.09 mmol) were added to a solution of 2-bromo-3,6-difluoropyridine (300 mg, 1.55 mmol) in DMF (3 mL) at rt and the reaction mixture was stirred at 90° C. for 14 h. The reaction mixture was cooled to rt, water (15 mL) was added, and the mixture was extracted with EtOAc (2×10 mL). The combined organic layer was washed with brine (15 mL), dried over Na₂SO₄ and concentrated. The crude product was purified by preparative HPLC, PrepMethod A (gradient: 10-50%), to give the title compound (300 mg, 58%); ¹H NMR (400 MHz, CDCl₃) δ 7.5 (m, 1H), 7.4 (m, 1H) 7.15 (m, 2H), 6.73 (m, 1H), 5.7 (s, 2H).

### Intermediate 43

### tert-Butyl (1R,5S,6r)-6-(6-((4-chloro-2-fluorobenzyl)oxy)-3-fluoropyridin-2-yl)-3-azabicyclo[3.1.0]hexane-3-carboxylate

The title compound was prepared from 2-bromo-6-((4-chloro-2-fluorobenzyl)oxy)-3-fluoropyridine Intermediate 42 (800 mg, 2.39 mmol) and tert-butyl (1R,5S,6s)-6-(4,4,5,5-tetramethyl-1,3,2-dioxaborolan-2-yl)-3-azabicyclo[3.1.0]hexane-3-carboxylate (739 mg, 2.39 mmol) in analogy with the description for Intermediate 2 The crude product was purified by preparative HPLC, PrepMethod A (isocratic 80%), to give the title compound (650 mg, 62%); MS (ESI) m/z [M+H]⁺ 437.2.

### Intermediate 44

### (1R,5S,6r)-6-(6-((4-Chloro-2-fluorobenzyl)oxy)-3-fluoropyridin-2-yl)-3-azabicyclo[3.1.0]hexane

The title compound was prepared from tert-butyl (1R,5S,6r)-6-(6-((4-chloro-2-fluorobenzyl)oxy)-3-fluoropyridin-2-yl)-3-azabicyclo[3.1.0]hexane-3-carboxylate Intermediate 43 (550 mg, 1.26 mmol) in analogy with the description for Intermediate 32 to give the HCl salt of the title compound (350 mg, 74%); MS (ESI) m/z [M+H]⁺ 337.0.

### Intermediate 45

### (1R,5S,6r)-6-(6-((4-Chloro-2-fluorobenzyl)oxy)-3-fluoropyridin-2-yl)-3-azabicyclo[3.1.0]hexane

The title compound was prepared from tert-butyl (1R,5S,6r)-6-(6-((4-chloro-2-fluorobenzyl)oxy)-3-fluoropyridin-2-yl)-3-azabicyclo[3.1.0]hexane-3-carboxylate Intermediate 43 (230 mg, 0.53 mmol) in analogy with the description for Intermediate 33 to give the TFA salt of the title compound (340 mg, 95%); MS (ESI) m/z [M+H]⁺ 337.0.

### Intermediate 46

### Methyl 2-(((1R,5S,6S)-6-(6-((4-chloro-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylate

The title compound was prepared from (1R,5S,6r)-6-(6-((4-Chloro-2-fluorobenzyl)oxy)-3-fluoropyridin-2-yl)-3-azabicyclo[3.1.0]hexane HCl Intermediate 44 (350 mg, 0.938 mmol) and methyl (S)-2-(chloromethyl)-1-(oxetan-2-ylmethyl)-1H-benzo[d]imidazole-6-carboxylate (332 mg, 1.13 mmol) in analogy with the description for Intermediate 12, except the reaction mixture was stirred at 50° C. at 14 h, to give the title compound (410 mg, 73%); MS (ESI) m z [M+H]⁺ 595.2.

### Intermediate 47

### Methyl 2-(((1R,5S,6r)-6-(6-((4-chloro-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylate

The title compound was prepared from (1R,5S,6r)-6-(6-((4-chloro-2-fluorobenzyl)oxy)-3-fluoropyridin-2-yl)-3-azabicyclo[3.1.0]hexane TFA Intermediate 45 (281 mg, 0.41 mmol) and methyl 2-(chloromethyl)-1-((1-ethyl-1H-imidazol-5-yl)methyl)-4-methoxy-1H-benzo[d]imidazole-6-carboxylate Intermediate 21 (100 mg, 0.28 mmol) in analogy with the description for Intermediate 12, except the reaction mixture was heated at 80° C. for 16 h. The reaction mixture was diluted with water (100 mL) and extracted with EtOAc (3×75 mL). The combined organic layer was dried over Na₂SO₄, filtered and evaporated. The crude product was purified by preparative TLC (DCM:MeOH, 10:1) to give the title compound (120 mg, 66%) as a white solid; MS (ESI) m/z [M+H]⁺ 663.

### Intermediate 48

### 2-Bromo-3-chloro-6-((4-chloro-2-fluorobenzyl)oxy)pyridine

The title compound was prepared from 2-bromo-3-chloro-6-fluoropyridine (220 mg, 1.05 mmol) and (4-chloro-2-fluorophenyl)methanol (252 mg, 1.57 mmol) in analogy with the description for Intermediate 42, but using Cs₂CO₃ (852 mg, 2.61 mmol) instead of K₂CO₃ as base. The crude product was purified by preparative TLC (petroleum ether:EtOAc, 15:1) to give the title compound (315 mg, 86%); MS (ESI) m/z [M+H]⁺ 350.

### Intermediate 49

### (1R,5S,6r)-6-(3-Chloro-6-((4-chloro-2-fluorobenzyl)oxy)pyridin-2-yl)-3-azabicyclo[3.1.0]hexane

### Step a) tert-Butyl (1R,5S6r)-6-(3-chloro-6-((4-chloro-2-fluorobenzyl)oxy)pyridin-2-yl)-3-azabicyclo[3.1.0]hexane-3-carboxylate

K₂CO₃ (236 mg, 1.71 mmol) was added to a suspension of 2-bromo-3-chloro-6-((4-chloro-2-fluorobenzyl)oxy)pyridine Intermediate 48 (200 mg, 0.57 mmol), tert-butyl (1R,5S,6s)-6-(4,4,5,5-tetramethyl-1,3,2-dioxaborolan-2-yl)-3-azabicyclo[3.1.0]hexane-3-carboxylate (194 mg, 0.63 mmol) and Pd(dppf)Cl₂·DCM (46 mg, 0.06 mmol) in 1,4-dioxane (10 mL) and water (1 mL). The reaction mixture was heated at 80° C. for 3 h. The reaction mixture was diluted with DCM (100 mL) and washed with water (100 mL) and sat brine (50 mL). The organic layer was dried over Na₂SO₄, filtered and concentrated. The crude product was purified by preparative TLC (EtOAc:petroleum ether, 1:5) to give the sub-title compound (173 mg, 67%) as a colorless gum.

### Step b) (1R,5S6r)-6-(3-Chloro-6-(4-chloro-2-fluorobenzyl)oxy)pyridin-2-yl)-3-azabicyclo[3.1.0]hexane

TFA (2 mL) was added to a solution of tert-butyl (1R,5S,6r)-6-(3-chloro-6-((4-chloro-2-fluorobenzyl)oxy)pyridin-2-yl)-3-azabicyclo[3.1.0]hexane-3-carboxylate Intermediate 49 Step a) (200 mg, 0.44 mmol) and the reaction mixture was stirred at rt for 15 h. The reaction mixture was concentrated at reduced pressure and the crude product was dried in vacuo to give the TFA salt of the title compound (240 mg, 68%) as a brown solid.

### Intermediate 50

### Methyl 2-(((1R,5S,6S)-6-(3-chloro-6-((4-chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylate

Methyl (S)-2-(chloromethyl)-4-methoxy-1-(oxetan-2-ylmethyl)-1H-benzo[d]imidazole-6-carboxylate Intermediate 6 (123 mg, 0.38 mmol) was added to a mixture of (1R,5S,6r)-6-(3-chloro-6-((4-chloro-2-fluorobenzyl)oxy)pyridin-2-yl)-3-azabicyclo[3.1.0]hexane TFA Intermediate 49 (220 mg, 0.38 mmol) and K₂CO₃ (210 mg, 1.52 mmol) in MeCN (2 mL) and the reaction mixture was stirred at 60° C. for 15 h. The reaction mixture was concentrated at reduced pressure, dissolved in DCM, and washed with water and sat brine. The organic layer was dried over Na₂SO₄, filtered and evaporated. The crude product was purified by preparative TLC (EtOAc:petroleum ether, 3:1) to give the title compound (227 mg, 93%) as a colorless oil which solidified on standing; MS (ESI) m/z [M+H]⁺ 641.2.

### Intermediate 51

### 2-Bromo-6-((4-chloro-2-fluorobenzyl)oxy)-3-methylpyridine

The title compound was prepared from (4-chloro-2-fluorophenyl)methanol (255 mg, 1.59 mmol) and 2-bromo-6-fluoro-3-methylpyridine (300 mg, 1.59 mmol) in analogy with the description for Intermediate 48. The crude product was purified by preparative TLC (petroleum ether:EtOAc, 5:1) to give the title compound (450 mg, 85%); MS (ESI) m z [M+H]⁺ 330/332.

### Intermediate 52

### (1R,5S,6r)-6-(6-((4-Chloro-2-fluorobenzyl)oxy)-3-methylpyridin-2-yl)-3-azabicyclo[3.1.0]hexane

The title compound was prepared in two steps from 2-bromo-6-((4-chloro-2-fluorobenzyl)oxy)-3-methylpyridine Intermediate 51 (200 mg, 0.60 mmol) and tert-butyl (1R,5S,6s)-6-(4,4,5,5-tetramethyl-1,3,2-dioxaborolan-2-yl)-3-azabicyclo[3.1.0]hexane-3-carboxylate (206 mg, 0.66 mmol) in analogy with the description for Intermediate 49 to give the TFA salt of the title compound (270 mg, 47%); MS (ESI) m/z [M+H]⁺ 333.

### Intermediate 53

### Methyl 2-(((1R,5S,6S)-6-(6-((4-chloro-2-fluorobenzyl)oxy)-3-methylpyridin-2-yl)-3-azabicyclo[3.1.0]hexan-3-yl)methyl)-4-methoxy-1-(((S)-oxetan-2-yl)methyl)-1H-benzo[d]imidazole-6-carboxylate

The title compound was prepared from (1R,5S,6r)-6-(6-((4-chloro-2-fluorobenzyl)oxy)-3-methylpyridin-2-yl)-3-azabicyclo[3.1.0]hexane Intermediate 52 (250 mg, 0.37 mmol) and methyl (S)-2-(chloromethyl)-4-methoxy-1-(oxetan-2-ylmethyl)-1H-benzo[d]imidazole-6-carboxylate Intermediate 6 (121 mg, 0.37 mmol) in analogy with the description for Intermediate 50 to give the title compound (278 mg, 64%) as a colorless oil which solidified on standing; MS (ESI) m/z [M+H]⁺ 621.2.

### Intermediate 54

### (1R,5S,6r)-6-(6-(Benzyloxy)pyridin-2-yl)-3-azabicyclo[3.1.0]hexane

The title compound was prepared in two steps from 2-(benzyloxy)-6-bromopyridine (500 mg, 1.89 mmol) and tert-butyl (1R,5S,6s)-6-(4,4,5,5-tetramethyl-1,3,2-dioxaborolan-2-yl)-3-azabicyclo[3.1.0]hexane-3-carboxylate (702 mg, 2.27 mmol) in analogy with the description for Intermediate 49 to give the TFA salt of the title compound (96% yield) as a yellow solid; MS (ESI) m/z [M+H]⁺ 267.

### Intermediate 55

### Methyl 2-(((1R,5S,6S)-6-(6-(benzyloxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylate

The title compound was prepared from (1R,5S,6r)-6-(6-(benzyloxy)pyridin-2-yl)-3-azabicyclo[3.1.0]hexane Intermediate 54 (0.82 g, 3.08 mmol) and methyl (S)-2-(chloromethyl)-4-methoxy-1-(oxetan-2-ylmethyl)-1H-benzo[d]imidazole-6-carboxylate Intermediate 6 (1.0 g, 3.08 mmol) in analogy with Intermediate 8 except for using DMF (20 mL) as solvent and heating the reaction mixture at 50° C. for 16 h. The crude product was purified by preparative TLC (EtOAc:petroleum ether, 4:1) to give the title compound (1.5 g, 88%) as a yellow solid; MS (ESI) m/z [M+H]⁺ 555.

### Intermediate 56

### Methyl 2-(((1R,5S,6S)-6-(6-hydroxypyridin-2-yl)-3-azabicyclo[3.1.0]hexan-3-yl)methyl)-4-methoxy-1-(((S)-oxetan-2-yl)methyl)-1H-benzo[d]imidazole-6-carboxylate

Pd/C (0.10 g, 0.94 mmol) was added to a solution of methyl 2-(((1R,5S,6S)-6-(6-(benzyloxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylate Intermediate 55 (1.00 g, 1.91 mmol) in EtOH (20 mL). The reaction mixture was stirred at −10° C. for 30 min and then hydrogenated at 30° C. for 3 h. The solids were filtered off and the resulting mixture was concentrated in vacuo. The residue was purified by straight phase chromatography on silica (EtOAc:petroleum ether, 1:10) to give the title compound (0.8 g, 90%) as a light yellow solid; MS (ESI) m/z [M+H]⁺ 465.

### EXAMPLES

### Example 1

### 2-(((1R,5S,6S)-6-(6-((4-Cyano-2-fluorobenzyl)oxy)-3-fluoropyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid

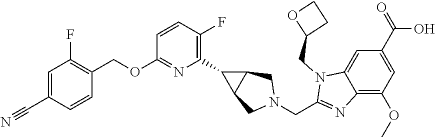 [SMILES: COc1cc(C(=O)O)cc2c1nc(CN1C[C@@H]3[C@H](C1)[C@H]3c1nc(OCc3ccc(C#N)cc3F)ccc1F)n2C[C@@H]1CCO1]

A solution of LiOH monohydrate (33 mg, 785 μmol) in H₂O (4 mL) was added to a solution of methyl 2-(((1R,5S,6S)-6-(6-((4-cyano-2-fluorobenzyl)oxy)-3-fluoropyridin-2-yl)-3-azabicyclo[3.1.0]hexan-3-yl)methyl)-4-methoxy-1-(((S)-oxetan-2-yl)methyl)-1H-benzo[d]imidazole-6-carboxylate Intermediate 7 (241 mg, 391 μmol) in THE (8 mL) and the reaction mixture was stirred at 0° C. to rt for 16 h. The mixture was concentrated under reduced pressure and the crude product was purified by preparative HPLC, PrepMethod A, (gradient: 40-65%) to give the title compound (88 mg, 37%); HRMS (ESI) m/z [M+H]⁺ calcd for C₃₂H₃₀F₂N₅O₅: 602.2210, found: 602.2201; ¹H NMR (400 MHz, DMSO-d₆) δ 12.78 (s, 1H), 7.93-7.84 (m, 2H), 7.71 (dd, 1H), 7.65 (t, 1H), 7.57 (t, 1H), 7.25 (d, 1H), 6.66 (dd, 1H), 5.38 (s, 2H), 5.04 (qd, 1H), 4.69 (dd, 1H), 4.60-4.42 (m, 2H), 4.35 (dt, 1H), 4.07 (d, 1H), 3.95 (s, 3H), 3.90 (d, 1H), 3.08 (d, 1H), 2.93 (d, 1H), 2.74-2.62 (m, 2H), 2.59 (d, 1H), 2.42 (qd, 2H), 1.93 (s, 2H).

### Example 2

### 2-(((1R,5S,6S)-6-(6-((4-Cyano-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylic acid

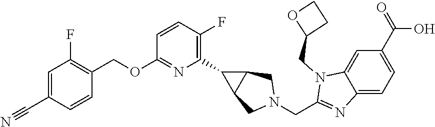 [SMILES: N#Cc1ccc(COc2ccc(F)c([C@H]3[C@@H]4CN(Cc5nc6ccc(C(=O)O)cc6n5C[C@@H]5CCO5)C[C@@H]43)n2)c(F)c1]

1,3,4,6,7,8-Hexahydro-2H-pyrimido(1,2-a)-pyrimidine (0.029 g, 0.20 mmol) was added in one portion to a solution of methyl 2-(((1R,5S,6S)-6-(6-((4-cyano-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylate Intermediate 8 (0.10 g, 0.17 mmol) in a mixture of MeCN (3 mL) and water (1 mL), and the reaction mixture was stirred at 20° C. for 16 h. The pH of the reaction mixture was adjusted to 4 with 0.5 M citric acid (aq). The reaction mixture was diluted with brine (25 mL) and extracted with EtOAc (3×50 mL). The combined organic layer was dried over Na₂SO₄, filtered and evaporated at reduced pressure. The crude product was purified by preparative HPLC, PrepMethod D, (gradient: 25-50%) to give the title compound (0.070 g, 61%) as a white solid; HRMS (ESI) m/z [M+H]f calcd for C₃₁H₂₈F₂N₅O₄: 572.2104, found: 572.2136; ¹H NMR (300 MHz, CD₃OD) δ 2.32 (s, 2H), 2.50 (t, 1H), 2.63 (s, 1H), 2.73-2.90 (m, 1H), 3.65 (s, 2H), 3.81 (d, 2H), 4.46 (dt, 1H), 4.58-4.83 (m, 5H), 5.22 (q, 1H), 5.44 (s, 2H), 6.69 (dd, 1H), 7.47 (t, 1H), 7.52-7.69 (m, 3H), 7.79 (d, 1H), 8.04 (dd, 1H), 8.35 (d, 1H).

### Example 3

### 2-(((1R,5S,6S)-6-(6-((4-Cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid

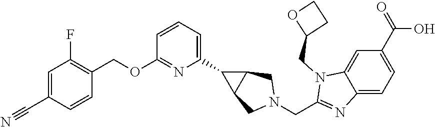 [SMILES: N#Cc1ccc(COc2cccc([C@H]3[C@@H]4CN(Cc5nc6ccc(C(=O)O)cc6n5C[C@@H]5CCO5)C[C@@H]43)n2)c(F)c1]

1,3,4,6,7,8-Hexahydro-2H-pyrimido(1,2-a)-pyrimidine (98 mg, 0.70 mmol) was added to a solution of methyl 2-(((1R,5S,6S)-6-(6-((4-cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylate Intermediate 11 (200 mg, 0.35 mmol) in a mixture of MeCN (2 mL) and water (0.5 mL) at 25° C., and the reaction mixture was stirred at 25° C. for 16 h. The pH of the reaction mixture was adjusted to 6 with 0.5 M citric acid. The reaction mixture was concentrated at reduced pressure and diluted with EtOAc (50 mL). The organic phase was washed with water (50 mL), and dried over Na₂SO₄, filtered and evaporated at reduced pressure. The crude product was purified by preparative HPLC, PrepMethod E, (gradient: 25-35%), to give the title compound (58 mg, 30%) as a white solid; HRMS (ESI) m/z [M+H]⁺ calcd for C₃₁H₂₉FN₅O₄: 554.2198, found: 554.2196; ¹H NMR (300 MHz, CD₃OD) δ 1.86-1.95 (m, 2H), 2.31 (t, 1H), 2.47-2.59 (m, 1H), 2.59-2.78 (m, 2H), 2.78-2.93 (m, 1H), 3.05 (d, 1H), 3.15 (d, 1H), 4.05 (d, 1H), 4.17 (d, 1H), 4.48 (dt, 1H), 4.60-4.76 (m, 2H), 4.79-4.92 (m, 1H), 5.18-5.30 (m, 1H), 5.47 (s, 2H), 6.60 (dd, 1H), 6.83 (dd, 1H), 7.46-7.73 (m, 5H), 7.99 (dd, 1H), 8.34 (dd, 1H).

### Example 4

### 2-(((1R,5S,6S)-6-(6-((4-Cyano-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid

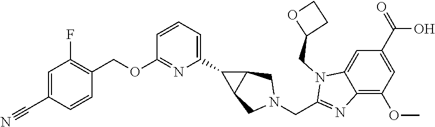 [SMILES: COc1cc(C(=O)O)cc2c1nc(CN1C[C@@H]3[C@H](C1)[C@H]3c1cccc(OCc3ccc(C#N)cc3F)n1)n2C[C@@H]1CCO1]

A solution of LiOH monohydrate (26 mg, 610 μmol) in water (2 mL) was added to a solution of methyl 2-(((1R,5S,6S)-6-(6-((4-cyano-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylate Intermediate 12 (135 mg, 234 μmol) in THE (4 mL) and the reaction mixture was stirred at 0° C.—rt for 16 h. Water (15 mL) was added to the reaction mixture and the pH was adjusted to 5-6 with NaHSO₄ (aq). The aqueous phase was extracted with EtOAc (5×15 mL), and the combined organic layer was dried over Na₂SO₄, filtered and concentrated under reduced pressure. The crude product was purified by preparative HPLC, PrepMethod A, (gradient: 30-65%) to give the title compound (32 mg, 25%); MS (ESI) m z [M+H]⁺ 584.2; ¹H NMR (500 MHz, DMSO-d₆) δ 7.87 (t, 2H), 7.66 (dt, 2H), 7.53 (t, 1H), 7.23 (s, 1H), 6.85 (d, 1H), 6.60 (d, 1H), 5.39 (s, 2H), 5.01 (dd, 1H), 4.68 (dd, 1H), 4.55 (dd, 1H), 4.46 (t, 1H), 4.39-4.25 (m, 1H), 4.01 (d, 1H), 3.92 (d, 4H), 3.02 (d, 1H), 2.91 (d, 1H), 2.69 (dd, 1H), 2.58 (dd, 2H), 2.37 (d, 1H), 2.20 (d, 1H), 1.83 (d, 2H).

### Example 5

### 2-(((1R,5S,6S)-6-(6-((4-Cyano-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid

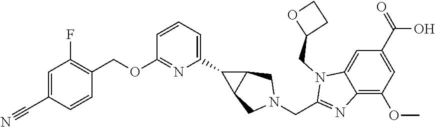 [SMILES: COc1cc(C(=O)O)cc2c1nc(CN1C[C@@H]3[C@H](C1)[C@H]3c1cccc(OCc3ccc(C#N)cc3F)n1)n2C[C@@H]1CCO1]

LiOH monohydrate (54 mg, 1.28 mmol) was added to a solution of methyl 2-(((1R,5S,6S)-6-(6-((4-cyano-2-fluorobenzyl)oxy)pyridin-2-yl)-3-azabicyclo[3.1.0]hexan-3-yl)methyl)-5-fluoro-1-(((S)-oxetan-2-yl)methyl)-1H-benzo[d]imidazole-6-carboxylate Intermediate 13 (300 mg, 512 μmol) in a mixture of THE and water (2:1, 5 mL) and the reaction mixture was stirred at rt for 16 h. The reaction mixture was acidified using NaH₂PO₄ (aq), diluted with water and the aqueous layer was extracted with EtOAc. The organic layer was dried over Na₂SO₄ and concentrated at reduced pressure. The residue was purified by preparative HPLC, PrepMethod F, (gradient: 0-50%), to give the title compound (170 mg, 58%); HRMS (ESI) m/z [M+H]⁺ calcd for C₃₁H₂₈F₂N₅O₄: 572.2104, found: 572.2116; ¹H NMR (600 MHz, DMSO-d₆) δ 8.09 (d, 1H), 7.86 (d, 1H), 7.70-7.66 (m, 1H), 7.62 (t, 1H), 7.52 (t, 1H), 7.39 (d, 1H), 6.84 (d, 1H), 6.59 (d, 1H), 5.38 (s, 2H), 5.00 (qd, 1H), 4.68 (dd, 1H), 4.55 (dd, 1H), 4.51-4.42 (m, 1H), 4.32 (dt, 1H), 4.02 (d, 1H), 3.88 (d, 1H), 3.04 (d, 1H), 2.92 (d, 1H), 2.76-2.66 (m, 1H), 2.66-2.52 (m, 3H), 2.43-2.34 (m, 2H), 2.19 (t, 1H), 1.82 (d, 2H).

### Example 6

### 2-(((1R,5S,6S)-6-(6-((4-Cyano-2-fluorobenzyl)oxy)pyridin-2-yl)-3-azabicyclo[3.1.0]hexan-3-yl)methyl)-1-(((S)-tetrahydrofuran-2-yl)methyl)-1H-benzo[d]imidazole-6-carboxylic acid

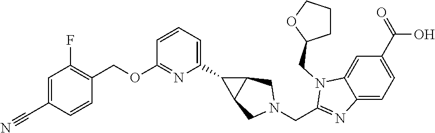 [SMILES: N#Cc1ccc(COc2cccc([C@H]3[C@@H]4CN(Cc5nc6ccc(C(=O)O)cc6n5C[C@@H]5CCCO5)C[C@@H]43)n2)c(F)c1]

A solution of methyl 2-(((1R,5S,6S)-6-(6-((4-cyano-2-fluorobenzyl)oxy)pyridin-2-yl)-3-azabicyclo[3.1.0]hexan-3-yl)methyl)-1-(((S)-tetrahydrofuran-2-yl)methyl)-1H-benzo[d]imidazole-6-carboxylate Intermediate 17 (163 mg, 0.28 mmol) and 1,3,4,6,7,8-hexahydro-2H-pyrimido[1,2-a]pyrimidine (81 mg, 0.58 mmol) in a mixture of MeCN (2 mL) and water (0.4 mL) was stirred at rt overnight. The reaction mixture was purified by preparative HPLC, PrepMethod C, (gradient: 25-65%), to give the title compound (93 mg, 58%) as a white powder; HRMS (ESI) m/z [M+H]⁺ calcd for C₃₂H₃₁FN₅O₄: 568.2354, found: 568.2338; ¹H NMR (500 MHz, DMSO-d₆) 1.57-1.72 (1H, m), 1.75-1.95 (4H, m), 2.02-2.15 (1H, m), 2.19 (1H, t), 2.59 (1H, dd), 2.67 (1H, dd), 2.92 (1H, d), 3.09 (1H, d), 3.63 (1H, q), 3.79 (1H, q), 3.88 (4H, m), 4.11 (1H, d), 4.18 (1H, qd), 4.41 (1H, dd), 4.53 (1H, dd), 5.41 (2H, s), 6.61 (1H, d), 6.83 (1H, d), 7.55 (1H, t), 7.61-7.68 (2H, m), 7.70 (1H, dd), 7.80 (1H, dd), 7.89 (1H, dd), 8.24 (1H, s).

### Example 7

### 2-(((1R,5S,6r)-6-(6-((4-Cyano-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylic acid

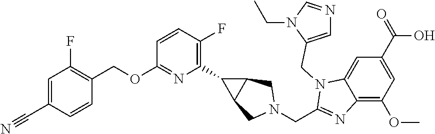 [SMILES: CCn1cncc1Cn1c(CN2C[C@@H]3[C@H](C2)[C@H]3c2nc(OCc3ccc(C#N)cc3F)ccc2F)nc2c(OC)cc(C(=O)O)cc21]

The title compound was prepared from methyl 2-(((1R,5S,6r)-6-(6-((4-cyano-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylate Intermediate 22 (24.5 mg, 0.37 mmol) as described in Example 1 to give the lithium salt of the title compound (10 mg, 45%); HRMS (ESI) m/z [M+H]⁺ calcd for C₃₄H₃₂F₂N₇O₄: 640.2478, found: 640.2506; ¹H NMR (600 MHz, DMSO-d₆) δ 7.91-7.82 (m, 1H), 7.70-7.66 (m, 1H), 7.65-7.57 (m, 2H), 7.56-7.51 (m, 1H), 7.30 (s, 1H), 6.63 (dd, 1H), 6.28 (s, 1H), 5.52 (s, 2H), 5.36 (d, 2H), 3.98 (q, 2H), 3.89-3.81 (m, 5H), 2.93 (d, 2H), 2.54-2.50 (m, partially overlapped with solvent), 2.13 (s, 1H), 1.86 (s, 2H), 1.18 (t, 3H).

### Example 8

### 4-Chloro-2-(((1R,5S,6S)-6-(6-((4-cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid

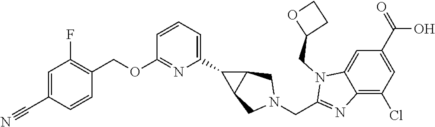 [SMILES: N#Cc1ccc(COc2cccc([C@H]3[C@@H]4CN(Cc5nc6c(Cl)cc(C(=O)O)cc6n5C[C@@H]5CCO5)C[C@@H]43)n2)c(F)c1]

The title compound was prepared from methyl 4-chloro-2-(((1R,5S,6S)-6-(6-((4-cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylate Intermediate 25 (358 mg, 0.595 mmol) in analogy with the description for Example 5 to give the title compound (34 mg, 10%); HRMS (ESI) m/z [M+H]⁺ calcd for C₃₁H₂₈ClFN₅O₄: 588.1808, found: 588.1814; ¹H NMR (600 MHz, DMSO-d₆) δ 8.20 (s, 1H), 7.87 (d, 1H), 7.77 (s, 1H), 7.74-7.60 (m, 2H), 7.54 (t, 1H), 6.86 (d, 1H), 6.60 (d, 1H), 5.40 (s, 2H), 5.04 (dt, 1H), 4.75 (dd, 1H), 4.65-4.57 (m, 1H), 4.48 (q, 1H), 4.34 (dt, 1H), 4.07 (d, 1H), 3.95 (d, 1H), 3.05 (d, 1H), 2.94 (d, 1H), 2.71 (p, 1H), 2.63 (d, 1H), 2.41 (q, 2H), 2.21 (d, 1H), 1.85 (d, 2H)

### Example 9

### 2-(((1R,5S,6S)-6-(6-((4-Cyano-2-fluorobenzyl)oxy)pyridin-2-yl)-3-azabicyclo[3.1.0]hexan-3-yl)methyl)-4-methyl-1-(((S)-oxetan-2-yl)methyl)-1H-benzo[d]imidazole-6-carboxylic acid

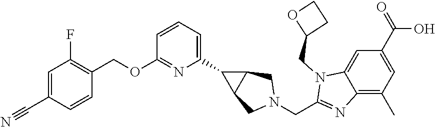 [SMILES: Cc1cc(C(=O)O)cc2c1nc(CN1C[C@@H]3[C@H](C1)[C@H]3c1cccc(OCc3ccc(C#N)cc3F)n1)n2C[C@@H]1CCO1]

The title compound was prepared from methyl 2-(((1R,5S,6S)-6-(6-((4-cyano-2-fluorobenzyl)oxy)pyridin-2-yl)-3-azabicyclo[3.1.0]hexan-3-yl)methyl)-4-methyl-1-(((S)-oxetan-2-yl)methyl)-1H-benzo[d]imidazole-6-carboxylate Intermediate 28 (50 mg, 0.860 mmol) in analogy with the description for Example 5 to give the title compound (35 mg, 80%); HRMS (ESI) m/z [M+H]⁺ calcd for C₃₂H₃₁FN₅O₄: 568.2354, found: 568.2374; ¹H NMR (600 MHz, DMSO-d₆) δ 8.04 (s, 1H), 7.87 (dd, 1H), 7.69 (dd, 1H), 7.64 (t, 1H), 7.60 (s, 1H), 7.53 (t, 1H), 6.85 (d, 1H), 6.60 (d, 1H), 5.40 (s, 2H), 5.02 (qd, 1H), 4.70 (dd, 1H), 4.57 (dd, 1H), 4.47 (td, 1H), 4.33 (dt, 1H), 4.05 (d, 1H), 3.93 (d, 1H), 3.04 (d, 1H), 2.93 (d, 1H), 2.76-2.67 (m, 1H), 2.63-2.55 (m, partly overlapping with solvent), 2.46-2.32 (m, partly overlapping with solvent), 2.21 (t, 1H), 1.84 (d, 2H).

### Example 10

### 2-(((1R,5S,6r)-6-(6-((4-Cyano-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylic acid

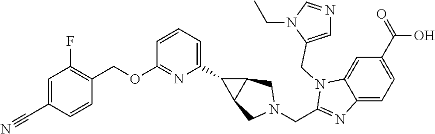 [SMILES: CCn1cncc1Cn1c(CN2C[C@@H]3[C@H](C2)[C@H]3c2cccc(OCc3ccc(C#N)cc3F)n2)nc2ccc(C(=O)O)cc21]

The title compound was prepared from methyl 2-(((1R,5S,6r)-6-(6-((4-cyano-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylate Intermediate 30 (250 mg, 0.413 mmol) in analogy with the description for Example 5 to give the title compound (140 mg, 60%); HRMS (ESI) m/z [M+H]⁺ calcd for C₃₃H₃₁FN₇O₃: 592.2468, found: 592.2470; ¹H NMR (400 MHz, DMSO-d₆) δ 8.05 (s, 1H), 7.88 (dd, 1H), 7.81 (dt, 1H), 7.76 (s, 1H), 7.73-7.60 (m, 3H), 7.53 (t, 1H), 6.74 (d, 1H), 6.59 (d, 1H), 6.28 (s, 1H), 5.65 (s, 2H), 5.39 (s, 2H), 4.04 (q, 2H), 3.96 (s, 2H), 2.92 (d, 2H), 2.56-2.51 (m, partly overlapping with solvent), 1.75 (s, 2H), 1.69 (d, 1H), 1.24 (t, 3H).

### Example 11

### 2-(((1R,5S,6S)-6-(6-((4-Chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid

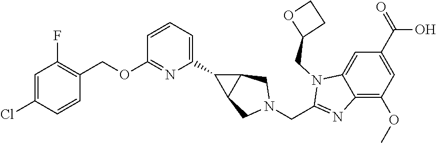 [SMILES: COc1cc(C(=O)O)cc2c1nc(CN1C[C@@H]3[C@H](C1)[C@H]3c1cccc(OCc3ccc(Cl)cc3F)n1)n2C[C@@H]1CCO1]

The title compound was prepared from methyl 2-(((1R,5S,6S)-6-(6-((4-chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylate Intermediate 34 (160 mg, 0.26 mmol) in analogy with the description for Example 2 to give the title compound (25 mg, 16%) as a white solid; HRMS (ESI) m/z [M+H]⁺ calcd for C₃₁H₃₁ClFN₄O₅: 593.1962, found: 593.1964; ¹H NMR (300 MHz, DMSO-d₆) 1.93 (2H, d), 2.25 (1H, t), 2.27-2.49 (partly overlapping with solvent, m), 2.54-2.80 (2H, m), 2.96 (1H, d), 3.07 (1H, d), 3.88-4.10 (5H, m), 4.29-4.39 (1H, m), 4.44-4.63 (2H, m), 4.65-4.76 (1H, m), 5.05 (1H, d), 5.33 (2H, s), 6.58 (1H, d), 6.86 (1H, d), 7.26 (1H, d), 7.31 (1H, dd), 7.44-7.58 (3H, m), 7.90 (1H, d), 12.86 (1H, s)

### Example 12

### 2-(((1R,5S,6r)-6-(6-((4-Chloro-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylic acid

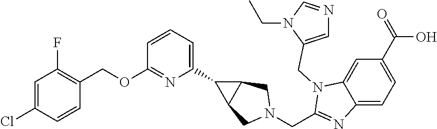 [SMILES: CCn1cncc1Cn1c(CN2C[C@@H]3[C@H](C2)[C@H]3c2cccc(OCc3ccc(Cl)cc3F)n2)nc2ccc(C(=O)O)cc21]

The title compound was prepared from methyl 2-(((1R,5S,6r)-6-(6-((4-chloro-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylate Intermediate 35 (154 mg, 0.238 mmol) in analogy with the description for Example 1 to give the title compound (104 mg, 69%); HRMS (ESI) m/z [M+H]⁺ calcd for C₃₂H₃₁ClFN₆O₃: 601.2124, found: 601.2172; ¹H NMR (600 MHz, DMSO-d₆) δ 7.91 (s, 1H), 7.77 (d, 1H), 7.72 (s, 1H), 7.53-7.38 (m, 4H), 7.27 (dd, 1H), 6.72 (d, 1H), 6.53 (d, 1H), 6.21 (s, 1H), 5.54 (s, 2H), 5.28 (s, 2H), 4.04 (q, 2H), 3.88 (s, 2H), 2.91 (d, 2H), 1.80 (d, 2H), 1.70 (t, 1H), 1.24 (t, 3H).

### Example 13

### 2-(((1R,5S,6S)-6-(6-((4-Chloro-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid

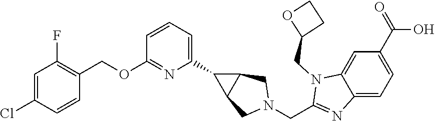 [SMILES: O=C(O)c1ccc2nc(CN3C[C@@H]4[C@H](C3)[C@H]4c3cccc(OCc4ccc(Cl)cc4F)n3)n(C[C@@H]3CCO3)c2c1]

The title compound was prepared from methyl 2-(((1R,5S,6S)-6-(6-((4-chloro-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylate Intermediate 36 (271 mg, 0.469 mmol) in analogy with the description for Example 1 to give the title compound (53 mg, 20%); MS (ESI) m/z [M+H]⁺ 563.0; ¹H NMR (400 MHz, CDCl₃) δ 9.2 (bs, 1H) 8.2 (s, 1H), 8.03 (d, 1H) 7.8 (d, 1H), 7.38 (m, 2H), 7.1 (m, 2H), 6.7 (d, 1H), 6.5 (d, 1H), 5.31 (s, 2H), 5.16 (m, 1H), 4.8-4.6 (m, 3H), 4.4 (m, 1H), 4.2 (m, 2H), 3.3-3.1 (m, 2H), 2.9-2.7 (m, 3H), 2.5-2.38 (m, 1H), 2.28 (s, 1H), 2.0 (s, 2H).

### Example 14

### 2-(((1R,5S,6r)-6-(6-((4-Chloro-2-fluorobenzyl)oxy)pyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylic acid

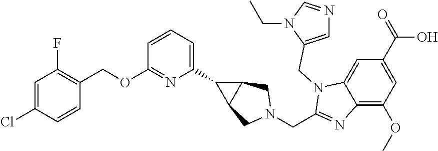 [SMILES: CCn1cncc1Cn1c(CN2C[C@@H]3[C@H](C2)[C@H]3c2cccc(OCc3ccc(Cl)cc3F)n2)nc2c(OC)cc(C(=O)O)cc21]

The title compound was prepared from methyl 2-(((1R,5S,6r)-6-(6-((4-chloro-2-fluorobenzyl)oxy)pyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylate Intermediate 37 (120 mg, 0.19 mmol) in analogy with the description for Example 2 to give the title compound (54 mg, 46%); HRMS (ESI) m/z [M+H]⁺ calcd for C₃₃H₃₃ClFN₆O₄: 631.2230, found: 631.2258; ¹H NMR (300 MHz, DMSO-d₆) 1.24 (3H, t), 1.72 (1H, t), 1.83 (2H, s), 2.55 (2H, d), 2.93 (2H, d), 3.96 (5H, d), 4.04 (2H, q), 5.30 (2H, s), 5.63 (2H, s), 6.29 (1H, d), 6.56 (1H, d), 6.75 (1H, d), 7.25-7.35 (2H, m), 7.42-7.58 (3H, m), 7.73 (2H, dd), 12.75 (1H, s).

### Example 15

### 2-(((1R,5S,6S)-6-(6-((4-Chloro-2-fluorobenzyl)oxy)pyridin-2-yl)-3-azabicyclo[3.1.0]hexan-3-yl)methyl)-4-fluoro-1-(((S)-oxetan-2-yl)methyl)-1H-benzo[d]imidazole-6-carboxylic acid

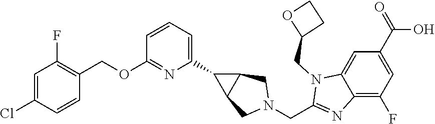 [SMILES: O=C(O)c1cc(F)c2nc(CN3C[C@@H]4[C@H](C3)[C@H]4c3cccc(OCc4ccc(Cl)cc4F)n3)n(C[C@@H]3CCO3)c2c1]

The title compound was prepared from methyl 2-(((1R,5S,6S)-6-(6-((4-chloro-2-fluorobenzyl)oxy)pyridin-2-yl)-3-azabicyclo[3.1.0]hexan-3-yl)methyl)-4-fluoro-1-(((S)-oxetan-2-yl)methyl)-1H-benzo[d]imidazole-6-carboxylate Intermediate 41 (140 mg, 0.24 mmol) in analogy with the description for Example 6. The reaction mixture was concentrated in vacuo and the crude product was purified by preparative HPLC, PrepMethod G (gradient: 34-48%) to give the title compound (40 mg, 29%); HRMS (ESI) m/z [M+H]⁺ calcd for C₃₀H₂₈ClF₂N₄O₄: 581.1762, found: 581.1756; ¹H NMR (300 MHz, DMSO-d₆) 1.93 (2H, s), 2.25 (1H, t), 2.44 (1H, t), 2.58-2.80 (3H, m), 2.98 (1H, d), 3.10 (1H, d), 3.96 (1H, d), 4.11 (1H, d), 4.37 (1H, dt), 4.51 (1H, q), 4.65 (1H, d), 4.79 (1H, dd), 5.06 (1H, d), 5.33 (2H, s), 6.58 (1H, d), 6.87 (1H, d), 7.31 (1H, dd), 7.44-7.58 (4H, m), 8.13-8.20 (1H, m). 13.08 (1H, s).

### Example 16

### 2-(((1R,5S,6S)-6-(6-((4-Chloro-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylic acid

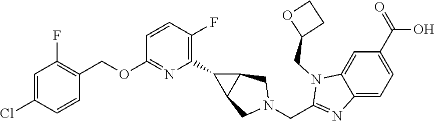 [SMILES: O=C(O)c1ccc2nc(CN3C[C@@H]4[C@H](C3)[C@H]4c3nc(OCc4ccc(Cl)cc4F)ccc3F)n(C[C@@H]3CCO3)c2c1]

The title compound was prepared from methyl 2-(((1R,5S,6S)-6-(6-((4-chloro-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylate Intermediate 46 (190 mg, 0.319 mmol) in analogy with the description for Example 4 to give the title compound (190 mg, 95%); HRMS (ESI) m/z [M+H]⁺ calcd for C₃₀H₂₈ClF₂N₄O₄: 581.1762, found: 581.1784; ¹H NMR (500 MHz, DMSO-d₆) δ 8.27 (s, 1H), 7.80 (d, 1H), 7.64 (d, 1H), 7.60-7.42 (m, 3H), 7.30 (d, 1H), 6.62 (dd, 1H), 5.28 (s, 2H), 5.15-4.96 (m, 1H), 4.73 (dd, 1H), 4.59 (d, 1H), 4.49 (q, 1H), 4.38 (q, 1H), 4.11 (d, 1H), 3.92 (d, 1H), 3.12 (d, 1H), 2.96 (d, 1H), 2.82-2.56 (m, 3H), 2.44-2.38 (m, partly overlapping with solvent), 2.00 (s, 2H).

### Example 17

### 2-(((1R,5S,6r)-6-(6-((4-Chloro-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylic acid

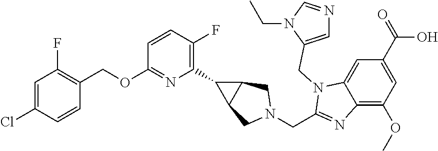 [SMILES: CCn1cncc1Cn1c(CN2C[C@@H]3[C@H](C2)[C@H]3c2nc(OCc3ccc(Cl)cc3F)ccc2F)nc2c(OC)cc(C(=O)O)cc21]

The title compound was prepared from methyl 2-(((1R,5S,6r)-6-(6-((4-chloro-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylate Intermediate 47 (110 mg, 0.17 mmol) in analogy with the description for Example 2. The crude product was purified by preparative HPLC, PrepMethod G (gradient: 27-399%), to give the title compound (42 mg, 38%) as a white solid; HRMS (ESI) m/z [M+H]⁺ calcd for C₃₃H₃₂ClF₂N₆O₄: 649.2136, found: 649.2164; ¹H NMR (300 MHz, DMSO-d₆) 1.18 (3H, t), 1.96 (2H, s), 2.18 (1H, d), 2.59 (2H, d), 2.99 (2H, d), 3.68-4.10 (7H, m), 5.28 (2H, s), 5.65 (2H, s), 6.39 (1H, s), 6.61 (1H, dd), 7.23-7.36 (2H, m), 7.42-7.60 (3H, m), 7.61-7.75 (2H, m).

### Example 18

### 2-(((1R,5S,6S)-6-(3-Chloro-6-((4-chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid

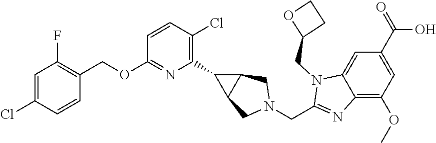 [SMILES: COc1cc(C(=O)O)cc2c1nc(CN1C[C@@H]3[C@H](C1)[C@H]3c1nc(OCc3ccc(Cl)cc3F)ccc1Cl)n2C[C@@H]1CCO1]

The title compound was prepared from methyl 2-(((1R,5S,6S)-6-(3-chloro-6-((4-chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylate Intermediate 50 (203 mg, 0.32 mmol) in analogy with the description for Example 6, except the reaction mixture was stirred at 40° C. for 18 h. The reaction mixture was concentrated and the crude product was purified by preparative HPLC, PrepMethod G (gradient 35-48%), to give the title compound (110 mg, 55%) as a white solid; HRMS (ESI) m/z [M+H]⁺ calcd for C₃₁H₃₀Cl₂FN₄O₅: 627.1572, found: 627.1616; ¹H NMR (300 MHz, DMSO-d₆) 2.02 (2H, q), 2.36-2.54 (partly overlapping with solvent, m), 2.58-2.78 (4H, m), 2.95 (1H, d), 3.11 (1H, d), 3.85-3.94 (1H, d), 3.96 (3H, s), 4.05-4.14 (1H, s), 4.32-4.42 (1H, m), 4.44-4.62 (2H, m), 4.67-4.72 (1H, m), 5.03-5.14 (1H, m), 5.32 (2H, s), 6.66 (1H, d), 7.27 (1H, d), 7.32 (1H, dd), 7.46-7.56 (2H, m), 7.71 (1H, d), 7.91 (1H, d).

### Example 19

### 2-(((1R,5S,6S)-6-(6-((4-Chloro-2-fluorobenzyl)oxy)-3-methylpyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid

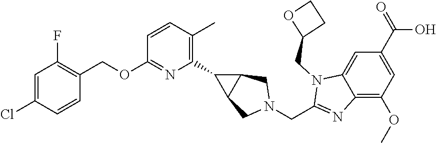 [SMILES: COc1cc(C(=O)O)cc2c1nc(CN1C[C@@H]3[C@H](C1)[C@H]3c1nc(OCc3ccc(Cl)cc3F)ccc1C)n2C[C@@H]1CCO1]

The title compound was prepared from methyl 2-(((1R,5S,6S)-6-(6-((4-chloro-2-fluorobenzyl)oxy)-3-methylpyridin-2-yl)-3-azabicyclo[3.1.0]hexan-3-yl)methyl)-4-methoxy-1-(((S)-oxetan-2-yl)methyl)-1H-benzo[d]imidazole-6-carboxylate Intermediate 53 (200 mg, 0.32 mmol) as described for Example 20 to give the title compound (61 mg, 31%); HRMS (ESI) m/z [M+H]⁺ calcd for C₃₂H₃₃ClFN₄O₅: 607.2118, found: 607.2126; ¹H NMR (300 MHz, DMSO-d6) 1.94 (2H, p), 2.29 (4H, d), 2.37-2.54 (partly overlapping with solvent, m), 2.56-2.57 (3H, m), 2.94 (1H, d), 3.07 (1H, d), 3.88-3.98 (4H, m), 4.08 (1H, d), 4.37 (1H, dt), 4.44-4.63 (1H, m), 4.54-4.64 (1H, m), 4.73 (1H, dd), 5.02-5.15 (1H, m), 5.30 (2H, s), 6.50 (1H, d), 7.17-7.33 (2H, m), 7.38-7.53 (3H, m), 7.90 (1H, d).

### Example 20-23

### General Synthesis Method A

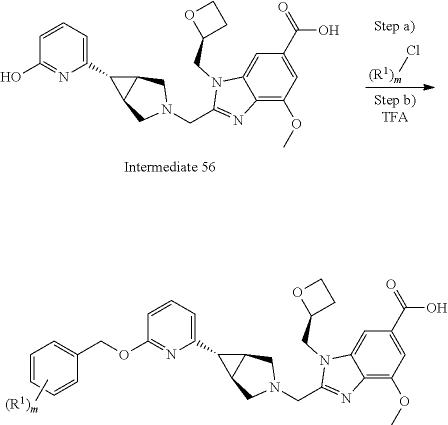

Step a) (R¹)ₘCl (0.17 mmol, 1.5 equiv) was added to a mixture of methyl 2-(((1R,5S,6S)-6-(6-hydroxypyridin-2-yl)-3-azabicyclo[3.1.0]hexan-3-yl)methyl)-4-methoxy-1-(((S)-oxetan-2-yl)methyl)-1H-benzo[d]imidazole-6-carboxylate Intermediate 56 (0.11 mmol, 1.0 equiv) and K₂CO₃ (0.33 mmol, 3.0 equiv) in DMF (2 mL). The reaction mixture was stirred at 40° C. for 16 h. The reaction mixture was diluted with water (4 mL) and extracted with DCM (3×2 mL). The combined organic layer was concentrated in vacuo.

Step b) THE (2 mL), LiOH (0.55 mmol, 5.0 equiv) and water (0.5 mL) was added to the crude product from Step a) and the reaction mixture was stirred at rt for 16 h. The reaction mixture was concentrated at reduced pressure, and the crude product was purified by preparative HPLC, PrepMethod H (gradient 5-90%), to give the title compound.

TABLE 4
The following Examples were prepared according to General Synthesis Method A from
Intermediate 56 and commercially available starting material ((R₁)ₘCl) where R₁ and m are
as defined in Claim 1, as described in Table 4 in the below.

| Ex- | | | |
|---|---|---|---|
| am- | | | MS (ESI) |
| ple | | | m/z |
| No | Structure | Name | [M + H]⁺ |
| 20 | 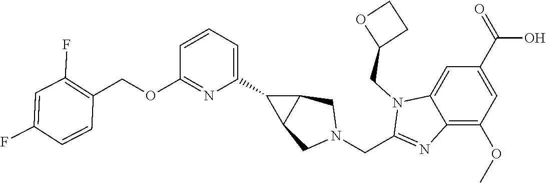 [SMILES: COc1cc(C(=O)O)cc2c1nc(CN1C[C@@H]3[C@H](C1)[C@H]3c1cccc(OCc3ccc(F)cc3F)n1)n2C[C@@H]1CCO1] | 2-(((1R,5S,6S)-6-(6-((2,4- Difluorobenzyl)oxy)pyridin-2-yl)- 3-azabicyclo[3.1.0]hexan-3- yl)methyl)-4-methoxy-1-(((S)- oxetan-2-yl)methyl)-1H- benzo[d]imidazole-6-carboxylic acid | 577.0 |
| 21 | 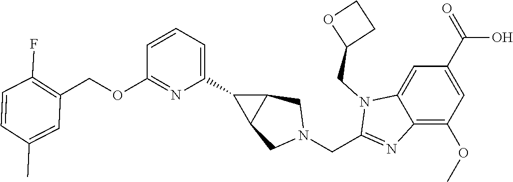 [SMILES: COc1cc(C(=O)O)cc2c1nc(CN1C[C@@H]3[C@H](C1)[C@H]3c1cccc(OCc3cc(C)ccc3F)n1)n2C[C@@H]1CCO1] | 2-(((1R,5S,6S)-6-(6-((2-Fluoro-5- methylbenzyl)oxy)pyridin-2-yl)- 3-azabicyclo[3.1.0]hexan-3- yl)methyl)-4-methoxy-1-(((S)- oxetan-2-y1)methyl)-1H- benzo[d]imidazole-6-carboxylic acid | 572.9 |
| 22 | 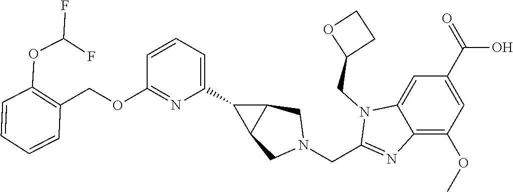 [SMILES: COc1cc(C(=O)O)cc2c1nc(CN1C[C@@H]3[C@H](C1)[C@H]3c1cccc(OCc3ccccc3OC(F)F)n1)n2C[C@@H]1CCO1] | 2-(((1R,5S,6S)-6-(6-((2- (Difluoromethoxy)benzyl)oxy)pyri- din-2-y1)-3- azabicyclo[3.1.0]hexan-3- yl)methyl)-4-methoxy-1-(((S)- oxetan-2-y1)methyl)-1H- benzo[d]imidazole-6-carboxylic acid | 606.9 |
| 23 | 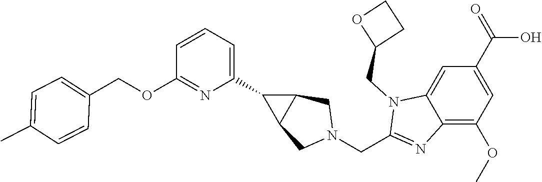 [SMILES: COc1cc(C(=O)O)cc2c1nc(CN1C[C@@H]3[C@H](C1)[C@H]3c1cccc(OCc3ccc(C)cc3)n1)n2C[C@@H]1CCO1] | 4-Methoxy-2-(((1R,5S,6S)-6-(6- ((4-methylbenzyl)oxy)pyridin-2- yl)-3-azabicyclo[3.1.0]hexan-3- yl)methyl)-1-(((S)-oxetan-2- yl)methyl)-1H- benzo[d]imidazole-6-carboxylic acid | 555.0 |

### Pharmacological Activity

CHOK1 GLP-1R cAMP Assay

A cell line stably expressing the human GLP-1R receptor (NM 002062.5, including the naturally-occurring variant Leu260Phe) in a CHO-K1 (ATCC® CCL-61™) was used for assay.

GLP-1 receptor mediated agonist activity was determined in a cell based assay measuring cyclic adenosine monophosphate (cAMP) levels in cells using Homogeneous Time-Resolved Fluorescence (HTRF) cAMP detection kit (CisBio catalog #62AM4PEC, cAMP Gs Dynamic range kit). The cAMP detection method is based on a competitive immunoassay, in which cAMP produced by the cells and cAMP labeled with the dye d2 compete for binding to a Europium-Cryptate-labeled anti-cAMP antibody. The specific HTRF signal is inversely proportional to the concentration of cAMP.

Compounds were added to individual well in 384 well-assay plates (Greiner #784076) using an Echo (LabCyte) dispenser from 10 mM stocks. Varying concentration of compounds were added to wells, and DMSO was used to normalize each well to a volume of 100 nL. A dose response curve of GLP1(7-36)NH2 (Bachem H-6795) was included in each run. 5 μL of cAMP concentration response standards are applied in specified wells in the assay plates. Cryo-preserved cells are thawed and resuspended in assay buffer pre-heated to 37° C. (20 mM HEPES pH 7.4, lx Hank's Balanced Salt Solution (HBSS, Life Technologies #14065) supplemented with 0.1% (w/v) bovine serum albumin (Sigma, A-7030). Cells were centrifuged at 250*g for 5 min at rt, and resuspended in room tempered assay buffer to a final density of 0.16*10⁶ cell/mL, to deliver 800 cells/well. 5 μL of assay buffer with 1 mM 3-isobutyl-1-methylxanthin (IBMX; Sigma cat I-7018) was dispensed per well in assay plates using a multidrop combi (Thermo Scientific) subsequently 5 μL of cell suspension was distributed to relevant wells in the assay using a multidrop dispenser. Assay plates were incubated 20 min at rt.

Detection reagents, Europium-Cryptate-labeled anti-cAMP antibody and cAMP labeled with the dye d2, are diluted in lysis buffer, provided by the manufacturer. 5 μL of each detection reagent is supplemented to each assay well using a multidrop dispenser. Assay plates are incubated in the dark for at least one h. The HTRF signal is measured using the HTRF module (excitation: 337 nm, emission A: 665 nm and emission B: 620 nm) in Pherastar FSX (BMG Labtech).

Raw data were converted to pM cAMP using the cAMP standard curve included in each run. Converted data were further analyzed in Genedata Screener (Genedata) and EC50 determinations were made from agonist dose-response curves analyzed with a curve fitting program using a 4-parameter logistic dose response equation (Equation y=A+((B−A)/1+((C/x)\hat{ }D))) where A is no stimulation, B is full stimulation, C is the EC50 and D is the Hill slope). The percent effect was determined relative to a saturating concentration of a full GLP-1R agonist (GLP1(7-36)NH2 has 100% effect in this assay setup).

The GLP-1R EC₅₀ values for the Example compounds are set forth in Table 1 herein below.

TABLE 1

| Ex. No. | GLP-1R EC50 (nM) |
|---|---|
| 1 | 7.7 |
| 2 | 30 |
| 3 | 75 |
| 4 | 12 |
| 5 | 240 |
| 6 | |
| 7 | 17 |
| 8 | 20 |
| 9 | 78 |
| 10 | 42 |
| 11 | 17 |
| 12 | 97 |
| 13 | 100 |
| 14 | 30 |
| 15 | 57 |
| 16 | 47 |
| 17 | 15 |
| 18 | 66 |
| 19 | 19 |
| 20 | 62 |
| 21 | 44 |
| 22 | 49 |
| 23 | 74 |

EndoC cAMP Accumulation Assay

A HTRF cAMP assay (cAMP Gs dynamic kit; CisoBio Cat #62AM4PEJ) was used to identify agonists of GLP-1R in a pancreatic insulinoma cell line (EndoC-βH1). The EndoC-βH1 cell line was sourced from Univercell Biosolutions and is a genetically engineered human pancreatic R cell line which exhibits glucose-inducible insulin secretion. EndoC-βH1 cells have detectable GLP-1R messenger ribonucleic acid (mRNA) as detected by quantitative polymerase chain reaction (qPCR). The functionality of GLP-1R signalling in EndoC-βH1 has been demonstrated by Exendin-4 treatment leading to augmented insulin secretion; an effect which is blunted with short hairpin ribonucleic acid (shRNA)-mediated knockdown of GLP-1R. The EndoC-βH1 cell line is a valid model of human beta cells and applicable for screenings to identify novel drug target candidates (Mol. Metab., 2018, 8, 144-157). CisBio HTRF cAMP kits are based on a competitive immunoassay using cryptate-labelled anti-cAMP antibody and d2-labeled cAMP. The detection kit is intended for the direct quantitative determination of cAMP. The specific signal (i.e. energy transfer) is inversely proportional to the concentration of cAMP in the standard or sample. Test compounds (10 mM in DMSO) were diluted into assay buffer (HBSS (Sigma #H8264) supplemented with 25 mM HEPES (Gibco #15630, pH 7.4), 0.1% BSA (Sigma #A3059) and 0.5 mM IBMX (Sigma #17018) included fresh on the day of the assay) into 96 well U-bottom plates (Greiner #650201). Diluted compounds were transferred to ECHO source polypropylene plates (Labcyte #P-05525) and dose response curves were dispensed acoustically using ECHO 550 into black shallow-well u-bottom 384-well HTRF Assay Plates (Corning 4514).

Cryovials of EndoC-H₁ (supplied at 1×10e⁷ cells/vial) were used directly for screening. The cryovials and were removed from N₂ (1) and thawed rapidly in a 37° C. water bath. The cells were resuspended in assay buffer and centrifuged at 300 g for 5 min. Cells were resuspended in assay buffer at the appropriate concentration, typically at 12e5 cells per mL (3000 cells per well, dependent on cell batch) and 2.5 μL diluted cells were added to all wells of destination plate by Multidrop combi reagent dispenser (Thermofisher). The plates were incubated at rt for 30 min. The assay was stopped by adding 2.5 μL anti-cAMP cryptate solution to all wells and 2.5 μL cAMP-d2 solution (both diluted 1:20 in lysis buffer) to columns 1-22 by Combi drop. A volume of 2.5 μL cAMP-d2 solution was added to wells E23 to P24 and 2.5 μL lysis buffer added to wells A23 to D24 by multichannel pipette. The plates were incubated at rt for 1 h and read on an Envision plate reader using excitation wavelength of 320 nm and emission of 590 nm and 660 nm.

Raw data from Envision is converted to % DeltaF according to the manufacturer's instructions. Dose response curves are analysed via 4-Parameter Logistical Analysis and assay plate Z′ values obtained. Samples are graphed as percentage (%) activation plots compared to GIP (1-42, Bachem H-5645) with assay window defined by negative control as basal cell cAMP levels and positive control are defined by maximum GIP (82.5 nM) signal. GLP1 (7-36 amide, Bachem H-6795) dose response curve was included on all plates.

The EndoC EC₅₀ values for the Example compounds are set forth in Table 2 herein below.

TABLE 2

| Ex. No. | EndoC EC50 (nM) |
|---|---|
| 1 | 5.0 |
| 2 | 26 |
| 3 | 21 |
| 4 | 5.8 |
| 5 | 200 |
| 6 | 290 |
| 7 | 38 |
| 8 | 10 |
| 9 | 56 |
| 10 | 88 |
| 11 | 6.5 |
| 12 | 150 |
| 13 | 23 |
| 14 | 7.4 |
| 15 | 28 |
| 16 | 24 |
| 17 | 9.3 |
| 18 | 42 |
| 19 | 9.6 |
| 20 | 43 |
| 21 | 1100 |
| 22 | 34 |
| 23 | 31 |

Inhibition of phosphodiesterase-3 (PDE3) has been shown to result in an increase in cardiovascular mortality in clinical trials (Movsesian M. A., Kukreja R. C. (2011) Phosphodiesterase Inhibition in Heart Failure. In: Francis S., Conti M., Houslay M. (eds) Phosphodiesterases as Drug Targets. Handbook of Experimental Pharmacology, vol 204. Springer, Berlin, Heidelberg. https:doi.org/10007/978-3-642-17969-3 10). Chronic treatment with PDE3 inhibitors has been shown to result in increased mortality, primarily as a result of arrhythmias and sudden death (Expert Opinion on Investigational Drugs, 2002, 11, 1529-1536; J. of Cardiovasc. Trans. Res., 2010, 3, 507-515) and it may therefore be an advantage to as far as possible avoid PDE3 inhibitory activity.

### PDE3 Assay

Evaluation of the effects of compounds on the activity of the human phosphodiesterase-3A is quantified by measuring the formation of 5′AMP from cAMP using a human recombinant enzyme expressed in a clonal isolate of Spodoptera frugiperda cells (Sf9) cells.

The test compound, reference compound or water (control) are added to a buffer containing 40 mM tris(hydroxymethyl)aminomethane (Tris)/HCl (pH 7.4) and 8 mM MgCl₂, 450 nMcAMP and 0.25 μCi [³H]cAMP.

Thereafter, the reaction is initiated by addition of the enzyme (about 1 U) and the mixture is incubated for 20 min at 22° C.

For basal control measurements, the enzyme is omitted from the reaction mixture. Following incubation SPA beads are added. After 30 min at 22° C. under shaking, the amount of [³H]⁺ 5′AMP is quantified with a scintillation counter (Topcount, Packard).

The results are expressed as a percent inhibition of the control enzyme activity. The standard inhibitory reference compound is milrinone (CAS number 78415-72-2), which is tested in each experiment at several concentrations to obtain an inhibition curve from which its IC₅₀ value is calculated.

The PDE3 IC₅₀ values for Example compounds and reference compounds are set forth in Table 3 herein below.

TABLE 3

| Ex. No. | PDE3 IC50 (μM) |
|---|---|
| 13 | >100 |
| 17 | >100 |
| 19 | >100 |
| Ref Comp A* | 4 |
| Ref Comp B** | 11 |
| Ref Comp C*** | 2 |
| Ref Comp D**** | 5 |

*Ref Comp A may be prepared as disclosed in WO2020103815, Ex 19
**Ref Comp B may be prepared as disclosed in WO2018109607, Ex 4A-01
***Ref Comp C may be prepared as disclosed in WO2021112538, Ex 73 or as disclosed in WO2021081207, Ex 67, or as disclosed in WO2020263695, Ex 3.
***Ref Comp D may be prepared as disclosed in WO2020263695, Ex 2

1. A compound of Formula (I), 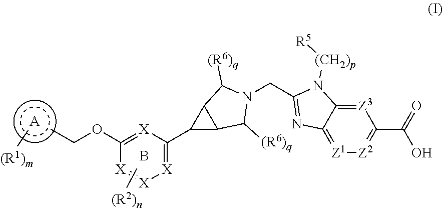
wherein
A is phenyl or pyridyl;
X is independently N or C, provided that no more than two atoms in the aromatic ring B are N;
Z¹ is N or CR³;
Z² and Z³ are each independently N or CR⁴, provided that when Z¹ or Z³ is N, Z² is CR⁴;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, 20° C. F₂H and OCF₃;
R⁵ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and where said cyclopropyl and cyclobutyl is substituted by 0 or 1 substituent selected from CN, OCH₃, OCFH₂, OCF₂H, OCF₃ and CH₂CN and 0, 1, 2 or 3 F;
R⁶ is independently selected from F, C₁₋₂alkyl and OC₁₋₂alkyl, wherein said C₁₋₂alkyl is substituted by 0, 1, 2 or 3 substituents independently selected from F;
m is 0, 1, 2 or 3;
n is 0 or 1;
p is 1, 2 or 3;
q is 0, 1 or 2;
or a pharmaceutically acceptable salt thereof.

2. A compound according to claim 1 of Formula (Ta) 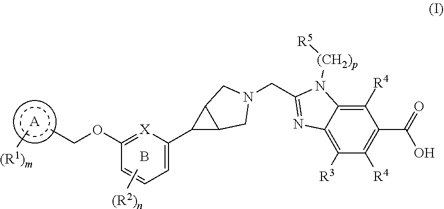
is wherein
A is phenyl or pyridyl;
X is N or C;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1, 2 or 3;
n is 0 or 1;
p is 1, 2 or 3;
q is 0, 1 or 2;
or a pharmaceutically acceptable salt thereof.

3. A compound according to claim 2,
wherein
A is phenyl;
X is N;
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1 or 2;
n is 0 or 1;
p is 1, 2 or 3;
or a pharmaceutically acceptable salt thereof.

4. A compound according to claim 1 selected from:
2-(((1R,5S,6S)-6-(6-((4-Cyano-2-fluorobenzyl)oxy)-3-fluoropyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylic acid,
is 2-(((1R,5S,6S)-6-(6-((4-Cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)pyridin-2-yl)-3-azabicyclo[3.1.0]hexan-3-yl)methyl)-1-(((S)-tetrahydrofuran-2-yl)methyl)-1H-benzo[d]imidazole-6-carboxylic acid,
2-(((1R,5S,6r)-6-(6-((4-Cyano-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylic acid,
4-Chloro-2-(((1R,5S,6S)-6-(6-((4-cyano-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid,
2-(((1R,5S,6S)-6-(6-((4-Cyano-2-fluorobenzyl)oxy)pyridin-2-yl)-3-azabicyclo[3.1.0]hexan-3-yl)methyl)-4-methyl-1-(((S)-oxetan-2-yl)methyl)-1H-benzo[d]imidazole-6-carboxylic acid,
2-(((1R,5S,6r)-6-(6-((4-Cyano-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylic acid,
2-(((1R,5S,6S)-6-(6-((4-Chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6r)-6-(6-((4-Chloro-2-fluorobenzyl)oxy)pyridin-2-yl)-3-azabicyclo[3.1.0]hexan-3-yl)methyl)-1-((1-ethyl-1H-imidazol-5-yl)methyl)-1H-benzo[d]imidazole-6-carboxylic acid,
2-(((1R,5S,6S)-6-(6-((4-Chloro-2-fluorobenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid,
2-(((1R,5S,6r)-6-(6-((4-Chloro-2-fluorobenzyl)oxy)pyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylic acid,
2-(((1R,5S,6S)-6-(6-((4-Chloro-2-fluorobenzyl)oxy)pyridin-2-yl)-3-azabicyclo[3.1.0]hexan-3-yl)methyl)-4-fluoro-1-(((S)-oxetan-2-yl)methyl)-1H-benzo[d]imidazole-6-carboxylic acid,
2-(((1R,5S,6S)-6-(6-((4-Chloro-2-fluorobenzyl)oxy)-3-fluoropyridin-2-yl)-3-azabicyclo[3.1.0]hexan-3-yl)methyl)-1-(((S)-oxetan-2-yl)methyl)-1H-benzo[d]imidazole-6-carboxylic acid,
is 2-(((1R,5S,6r)-6-(6-((4-Chloro-2-fluorobenzyl)oxy)-3-fluoropyridin-2-yl)-3-azabicyclo[3.1.0]hexan-3-yl)methyl)-1-((1-ethyl-1H-imidazol-5-yl)methyl)-4-methoxy-1H-benzo[d]imidazole-6-carboxylic acid,
2-(((1R,5S,6S)-6-(3-Chloro-6-((4-chloro-2-fluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((4-Chloro-2-fluorobenzyl)oxy)-3-methylpyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((2,4-Difluorobenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((2-Fluoro-5-methylbenzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
2-(((1R,5S,6S)-6-(6-((2-(Difluoromethoxy)benzyl)oxy)pyridin-2-yl)-3-azabicyclo[3.1.0]hexan-3-yl)methyl)-4-methoxy-1-(((S)-oxetan-2-yl)methyl)-1H-benzo[d]imidazole-6-carboxylic acid,
4-Methoxy-2-(((1R,5S,6S)-6-(6-((4-methylbenzyl)oxy)pyridin-2-yl)-3-azabicyclo[3.1.0]hexan-3-yl)methyl)-1-(((S)-oxetan-2-yl)methyl)-1H-benzo[d]imidazole-6-carboxylic acid,
or a pharmaceutically acceptable salt thereof.

5. A compound according to any of claims 1-4, or a pharmaceutically acceptable salt thereof, for use as a medicament.

6. A pharmaceutical composition comprising a compound according to claim 1, or a pharmaceutically acceptable salts thereof, optionally in admixture with a pharmaceutically acceptable adjuvant, diluents or carrier.

7. A method of treating, or reducing the risk of, cardiovascular disease or metabolic conditions which comprises administering to a person suffering from or at risk of, said disease or condition, a therapeutically effective amount of a compound according to claim 1, or a pharmaceutically acceptable salt thereof.

8. The method according to claim 1, wherein said disease is type 2 diabetes.

9. A compound according to claim 1, or a pharmaceutically acceptable salt thereof, for use as a medicament.

10. A compound according to claim 1, or a pharmaceutically acceptable salt thereof, for use as in the treatment of type 2 diabetes.

11. The use of a compound according to claim 1, or a pharmaceutically acceptable salt thereof, in the manufacture of a medicament, for the treatment or prophylaxis of cardiovascular disease or metabolic conditions.
